(19) United States
(12) Patent Application Publication
(10) Pub. No.: US 2018/0298317 A1
(43) Pub. Date: Oct. 18, 2018

(54) DEVICES FOR SIMULATING A FUNCTION OF A TISSUE AND METHODS OF USE AND MANUFACTURING THEREOF

(71) Applicant: PRESIDENT AND FELLOWS OF HARVARD COLLEGE, Cambridge, MA (US)

(72) Inventors: Donald E. INGBER, Boston, MA (US); Andries VAN DER MEER, Enschede (NL); Anna HERLAND, Cambridge, MA (US)

(21) Appl. No.: 15/568,515

(22) PCT Filed: Apr. 25, 2016
(86) PCT No.: PCT/US16/29164

§ 371 Date: Oct. 23, 2017

Related U.S. Application Data
(60) Provisional application No. 62/152,355, filed on Apr. 24, 2015.
(60) Provisional application No. 62/299,340, filed on Feb. 24, 2016.

(51) Int. Cl. C12M 3/06; C12M 1/12; C12M 1/00; C12M 1/42; C12N 5/079; G01N 33/50

(52) U.S. Cl. CPC C12M 23/16; C12M 25/14; C12M 29/10; C12M 35/08; C12N 2502/081; C12N 5/0618; C12N 5/0622; G01N 33/5005; C12M 25/02

(57) ABSTRACT

Systems and methods for producing and using a body having a first structure defining a first chamber, a second structure defining a second chamber, a membrane located at an interface region between the first chamber and the second chamber to separate the first chamber from the second chamber. The first chamber comprises a first permeable matrix disposed therein and the first permeable matrix comprises at least one or a plurality of lumens each extending therethrough, which is optionally lined with at least one layer of cells. The second chamber can comprise cells cultured therein. The systems and methods described herein can be used for various applications, including, e.g., growth and/or differentiation of primary cells, and/or simulation of a microenvironment in living tissues and/or organs (to model physiology or disease states, and/or to identify therapeutic agents). The systems and methods can also permit co-cultures of two or more different cell types.

### CROSS-REFERENCE TO RELATED APPLICATIONS

This application claims the benefit of priority to U.S. Provisional Patent Application No. 62/152,355, filed Apr. 24, 2015, and U.S. Provisional Application No. 62/299,340, filed Feb. 24, 2016, both of which are hereby incorporated by reference in their entireties.

### GOVERNMENT SUPPORT

This invention was made with government support under Grant No. W911NF-126-2-0036 awarded by DARPA. The government has certain rights in the invention.

### FIELD OF INVENTION

Embodiments of various aspects described herein relate generally to microfluidic devices and methods of use and manufacturing thereof. In some embodiments, the microfluidic devices can be used for culture and/or support of living cells such as mammalian cells, insect cells, plant cells, and microbial cells, and/or for simulating a function of a tissue.

### BACKGROUND

The blood-brain barrier is a physiological barrier that controls transport from blood to the brain and vice versa. One of the main players in maintaining the blood-brain barrier comprises the cerebral capillary endothelium, which limits passive transport from the blood by forming a monolayer with tight junctions and by actively pumping unwanted molecules back into the blood. In addition, the endothelium regulates the active transport of molecules and/or cells into the brain by receptor-mediated transcytosis.

The blood vessels in the brain are of major physiological importance because they maintain the blood-brain barrier (BBB), support molecular transport across this tight barrier, control local changes in oxygen and nutrients, and regulate the local immune response in the brain. Neurovascular dysfunction also has been linked to a wide spectrum of neurological disorders including multiple sclerosis, Alzheimer's disease, brain tumors, and the like. Due to its relevance for neurophysiology and pathophysiology, more realistic models of the human neurovascular niche are needed to advance fundamental and translational research, as well development of new and more effective therapeutics.

The BBB is formed by the continuous brain microvascular endothelium, its underlying basement membrane, pericytes that tightly encircle the endothelium, and astrocytes in the surrounding tissue space that extend their cell processes towards the endothelium and insert on the basement membrane. Together, these cells maintain a highly selective permeability barrier between the blood and the brain compartments that is critical for normal brain physiology. Importantly, the pericytes and astrocytes convey cues that are required for normal function and differentiation of the brain microvascular endothelium, and all three cell types—endothelial cells, pericytes, and astrocytes—are required for maintenance of the normal physiology of the neurovasculature and maintenance of BBB integrity in vivo as well as in vitro. Astrocytes also have been shown to display a large number of receptors involved in innate immunity, and when activated, to secrete soluble factors mediating both innate and adaptive immune responses. Brain pericytes have likewise been demonstrated to respond to inflammatory stimuli resulting in release of pro-inflammatory cytokines. However, the complex interaction between these cell types and the microvascular endothelium make it extremely difficult to analyze their individual contribution to neuroinflammation in vivo.

In addition to the endothelium being involved in maintaining the BBB, the endothelium can also rely on a direct cellular and/or acellular microenvironment to maintain differentiation and functionality. Some key factors in the cerebral endothelial microenvironment include, for example, cerebral pericytes, astrocytes, neurons, extracellular matrices, and combinations thereof. Together, these cells and biomolecules can form the neurovascular unit, which is a key organ subunit that is known to be important in neurological function and disease.

The blood-brain barrier is of major clinical relevance. Not only because dysfunction of the blood-brain barrier leads to degeneration of the neurovascular unit, but also because drugs that are supposed to treat neurological disorders often fail to permeate the blood-brain barrier. Because of its importance in disease and medical treatment, it would be highly advantageous to have a predictive model of the human blood-brain barrier that recapitulates significant aspects of the cerebral endothelial microenvironment in a controlled way.

Microfluidic device technology can be used to engineer models of human tissues and organs. Multiple microfluidic models of the blood-brain barrier have been previously reported, e.g., in Griep et al., Biomed Microdevices (2013) 15: 145-150; Achyuta et al. Lab Chip (2013) 13, 542-553; Booth and Kim, Lab Chip (2012) 12, 1784-1792; Yeon et al. Biomed Microdevices (2012) 14: 1141-1148. However, these existing models are lacking a controlled integration of the extracellular matrix, and a controlled and physiologically realistic three-dimensional endothelialized lumen. Accordingly, there is a need to engineer highly realistic models of human tissues and organs.

### SUMMARY

Aspects described herein stem from, at least in part, design of devices that allow for a controlled and physiologically realistic co-culture of one or more endothelialized lumens in one chamber with monolayers and/or three-dimensional cultures of tissue-specific cells in other chambers, where the chambers are aligned (e.g., vertically) with one another with one or more membranes separating them from one another. In one aspect, the inventors have used such devices to mimic the organization and/or function of a blood brain barrier in vitro. For example, the inventors have patterned a three-dimensional, endothelial cell-lined lumen, e.g., with generally circular cross-sectional geometries, through a first permeable matrix (e.g., extracellular matrix gel such as collagen) disposed in a first microchannel to mimic the structure of blood vessels in vitro, and also have populated a second microchannel that is separated from the first microchannel by a membrane, with astrocytes and/or neurons. In particular, in some embodiments, the astrocytes can be cultured on one side of the membrane facing the second microchannel, and neurons can be distributed in a second permeable matrix (e.g., extracellular matrix gel such as MATRIGEL® (Discovery Labware, Inc. (Bedford, Mass., USA)) that is disposed in the second microchannel. Not only does the first permeable matrix comprise an endothelial-lined lumen or a pericyte/endothelium-lined lumen extending therethrough, in some embodiments, the first permeable matrix can also comprise pericytes. Accordingly, the inventors, in one aspect, have developed a neurovascular co-culture with an organization that is highly reminiscent of the organization of the neurovascular unit in vivo—endothelial cells facing an open lumen, and interacting with a matrix (e.g., an extracellular matrix) comprising pericytes on their basal side, whereas a layer of astrocytes separates the perivascular gel from a neuronal compartment, in which neurons grow and interact to form a neuronal network. By choosing an appropriate matrix (e.g., an extracellular matrix) and geometries, neuronal and astrocytic cells can grow cellular processes that can penetrate the vascular and neuronal compartment, respectively. In addition, by culturing appropriate cell types in different compartments, the devices can be used to mimic organization and/or function of different tissues. Accordingly, embodiments of various aspects described herein relate to devices for simulating a function of a tissue and methods of making and using the same.

Some aspects described herein relate to devices for simulating a function of a tissue. The devices generally comprise (i) a first structure defining a first chamber, the first chamber comprising a first permeable matrix disposed therein, wherein the first permeable matrix comprises at least one or a plurality of (i.e., at least two or more, including, e.g., at least three or more) lumens each extending therethrough; (ii) a second structure defining a second chamber, the second chamber comprising cells disposed therein; and (iii) a membrane located at an interface region between the first chamber and the second chamber to separate the first chamber from the second chamber, the membrane including a first side facing toward the first chamber and a second side facing toward the second chamber. The cells disposed in the second chamber can be adhered on the second side of the membrane and/or distributed in a second permeable matrix disposed in the second chamber.

Thus, in one aspect described herein, a device for simulating a function of a tissue comprises: (i) a first structure defining a first chamber, the first chamber comprising a first permeable matrix disposed therein, wherein the first permeable matrix comprises at least one or a plurality of (i.e., at least two or more, including, e.g., at least three or more) lumens each extending therethrough; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the second side comprises cells of a first type adhered thereon.

In some embodiments, the cells of the first type adhering on the second side of the membrane can form a cell monolayer and/or a three-dimensional or stratified structure.

In some embodiments, the second chamber can comprise a second permeable matrix disposed therein. In some embodiments, the second permeable matrix can comprise cells of a second type.

In another aspect described herein, a device for simulating a function of a tissue, comprises: (i) a first structure defining a first chamber, the first chamber comprising a first permeable matrix disposed therein, wherein the first permeable matrix comprises at least one or a plurality of (i.e., at least two or more, including, e.g., at least three or more) lumens each extending therethrough; (ii) a second structure defining a second chamber, the second chamber comprising a second permeable matrix disposed therein; and (iii) a membrane located at an interface region between the first chamber and the second chamber to separate the first chamber from the second chamber, the membrane including a first side facing toward the first chamber and a second side facing toward the second chamber.

In some embodiments, the second side of the membrane can comprise cells of a first type adhered thereon.

In some embodiments of this aspect and other aspects described herein, the lumen(s) can be configured to mimic a duct or sinus of a tissue or an organ, a blood vessel, or the like. For example, in some embodiments, the lumen(s) can be lined with at least one layer of cells comprising blood vessel-associated cells and/or tissue-specific cells (e.g., tissue-specific epithelial cells). Examples of blood vessels-associated cells include, but are not limited to, endothelial cells, fibroblasts, smooth muscle cells, pericytes, and a combination of two or more thereof. In one embodiment, the lumen(s) can be lined with an endothelial cell monolayer. In some embodiments, the lumen(s) can be lined with pericytes (e.g., a sparse layer of pericytes) covered by an endothelial cell monolayer.

In some embodiments of this aspect and other aspects described herein, the second permeable matrix can comprise cells of a second type distributed therein.

In some embodiments of this aspect and other aspects described herein, the first permeable matrix can comprise cells of a third type distributed therein.

In some embodiments of this aspect and other aspects described herein, the first side of the membrane can comprise cells of a fourth type adhered thereon.

The cells of the first type, second type, third type, and/or fourth type can each independently comprise a type of tissue-specific cell. Appropriate tissue-specific cells can be selected depending on the organization and/or function of a tissue to be modeled. For example, tissue-specific cells are generally cells derived from a tissue or an organ including, e.g., but not limited to, a lung, a liver, a kidney, skin, an eye, a brain, a blood-brain-barrier, a heart, a gastrointestinal tract, airways, a reproductive organ, and a combination of two or more thereof

In some embodiments of this aspect and other aspects described herein, the second side of the membrane can comprise blood vessel-associated cells, including, but not limited to, endothelial cells and/or pericytes. In these embodiments, the lumen(s) can be lined with tissue-specific cells (e.g., ductal epithelial cells) to simulate a function of a duct or sinus of a tissue or an organ. In some embodiments, the first permeable matrix can comprise connective tissue cells embedded therein.

In some embodiments, the tissue-specific cells cultured in the devices described herein can comprise cells that are present in a cerebral endothelial microenvironment to mimic the organization, function, and/or physiology of a blood-brain-barrier. Accordingly, a further aspect described herein relates to a device for simulating a function of a blood-brain-barrier. Such devices comprise: (i) a first structure defining a first chamber, the first chamber comprising a first permeable matrix disposed therein, wherein the first permeable matrix comprises at least one or a plurality of (i.e., at least two or more, including, e.g., at least three or more) lumens each extending therethrough, and the lumen(s) is/are lined with at least one endothelial cell layer; (ii) a second structure defining a second chamber, the second chamber comprising a first type of brain microenvironment-associated cell distributed therein; and (iii) a membrane located at an interface region between the first chamber and the second chamber to separate the first chamber from the second chamber, the membrane comprising a first side facing toward the first chamber and a second side facing toward the second chamber.

In some embodiments, the first type of brain microenvironment-associated cell can be adhered on the second side of the membrane facing the second chamber. In some embodiments, the first type of brain microenvironment-associated cell can be embedded in a second permeable matrix disposed in the second chamber. Examples of the first type of brain microenvironment-associated cell include, but are not limited to astrocytes, microglia, neurons, and a combination of two or more thereof.

In some embodiments, the first permeable matrix can comprise a second type of brain microenvironment-associated cell distributed therein. Examples of the second type of brain microenvironment-associated cell include, but are not limited to, pericytes, astrocytes, microglia, fibroblasts, smooth muscle cells, or a combination of two or more thereof

In some embodiments, the lumen(s) can be lined with pericytes (e.g., a sparse layer of pericytes) covered by an endothelial cell monolayer.

In some embodiments of this aspect and other aspects described herein, the lumen(s) can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or crosslinking the viscous solution. Thus, one or a plurality of lumen(s) each extending through the first permeable matrix can be created.

In some embodiments of this aspect and other aspects described herein, the first and second permeable matrices can each independently comprise a hydrogel, an extracellular matrix gel, a polymer matrix, a monomer gel that can polymerize, a peptide gel, or a combination of two or more thereof In one embodiment, the first permeable matrix can comprise an extracellular matrix gel (e.g., collagen). In one embodiment, the second permeable matrix can comprise an extracellular matrix gel and/or a protein mixture gel representing an extracellular microenvironment (e.g., MATRIGEL®). In some embodiments, the first and the second permeable matrices can each independently comprise a polymer matrix. Any suitable method may be used to create permeable polymer matrices including, but not limited to, particle leaching from suspensions in a polymer solution, solvent evaporation from a polymer solution, solid-liquid phase separation, liquid-liquid phase separation, etching of specific “block domains” in block co-polymers, phase separation of block-copolymers, chemically cross-linked polymer networks with defined permeabilities, and a combination of two or more thereof

The first chamber and the second chamber of the devices described herein can have the same height or different heights. In some embodiments, the height of the first chamber can be higher than the height of the second chamber. For example, in some embodiments, the height of the first chamber can range from about 100 μm to about 50 mm, or about 200 μm to about 10 mm. In some embodiments, the height of the second chamber can range from 20 μm to about 1 mm, or about 50 μm to about 500 μm.

In some embodiments, the height of the first chamber and width of the first chamber can be configured to have a height: width ratio that accommodates the geometry of the lumen(s) and/or number of lumens to be arranged along the width and/or height of the first chamber. For example, for a circular cross-sectional lumen disposed in the first chamber, the height and width of the first chamber can be configured in a ratio of about 1:1. In some embodiments where at least two or more lumens are arranged side-by-side along the width of the first chamber, the height and width of the first chamber can be configured in a ratio less than 1:1 (i.e., the width of the first chamber is greater than the height of the first chamber), including, e.g., 1:2, 1:3, 1:4; 1:5; 1:6; 1:7; 1:8; 1:9; or 1:10. Thus, the width and/or height of the first chamber can increase with the number of lumens arranged along the width and/or height of the first chamber. In some embodiments, the height of the first chamber and the width of the first chamber can be configured to have a ratio of about 1:1 to about 1:6.

The membrane separating the first chamber and the second chamber in the devices described herein can be rigid or at least partially flexible. In some embodiments, the membrane can be configured to deform in a manner (e.g., stretching, retracting, compressing, twisting and/or waving) that simulates a physiological strain experienced by the cells in its native microenvironment. In these embodiments, the membrane can be at least partially flexible. In some embodiments, the membrane can be configured to provide a supporting structure to permit growth of a defined layer of cells thereon.

The membrane can be of any suitable thickness. In some embodiments, the membrane can have a thickness of about 1 μm to about 100 μm or about 100 nm to about 50 μm. In one embodiment, the membrane can have a thickness of about 50 μm.

The membrane can be non-porous or porous. In some embodiments where at least a portion of the membrane is porous, the pores can have a diameter of about 0.1 μm to about 15 μm.

The membrane can be fabricated from any biocompatible, biological, and/or biodegradable materials.

While the first chamber and the second chamber can be in any geometry or three-dimensional structure, in some embodiments, the first chamber and the second chamber can be configured to be form channels.

Methods of making a device for simulating a function of a tissue are also described herein. The method comprises: (a) providing a body comprising: (i) a first structure defining a first chamber, at least a portion of the first chamber filled with a viscous solution of first matrix molecules disposed therein, (ii) a second structure defining a second chamber; and (iii) a membrane located at an interface region between the first chamber and the second chamber to separate the first chamber from the second chamber, the membrane including a first side facing toward the first chamber and a second side facing toward the second chamber; (b) flowing at least one pressure-driven fluid with viscosity lower than that of the viscous solution through the viscous solution in the first chamber to create one or more lumens each extending through the viscous solution; (c) gelling, polymerizing and/or crosslinking the viscous solution in the first chamber, thereby forming a first permeable matrix comprising one or more lumen(s) each extending therethrough; and (d) populating at least a portion of the second chamber with tissue specific cells.

In some embodiments, the tissue specific cells of a first type can be populated on the second side of the membrane. In some embodiments, the tissue specific of a second type can be populated in a second permeable matrix disposed in the second chamber. Accordingly, in these embodiments, the method can further comprise forming a second permeable matrix in the second chamber, wherein the second permeable matrix comprises the tissue specific cells of a second type.

In some embodiments, the method can further comprise forming at least one layer of cells comprising blood vessel-associated cells on the inner surface of the lumen(s). In some embodiments, the inner surface of the lumen(s) can comprise an endothelial cell monolayer.

In some embodiments, the viscous solution filling the first chamber can comprise tissue specific cells of a third type.

Devices for simulating a function of a tissue produced by the methods of making the same are also provided herein.

The ability of the devices described herein to recapitulate a physiological microenvironment and/or function can provide an in vitro model versatile for various applications such as, but not limited to, modeling a tissue-specific physiological condition (e.g., normal and disease states), study of cytokine release, and/or identification of therapeutic agents. Accordingly, methods of using the devices are also described herein. In one aspect, the method comprises: (a) providing at least one device comprising: (i) a first structure defining a first chamber, the first chamber comprising a first permeable matrix disposed therein, wherein the first permeable matrix comprises at least one or a plurality of (i.e., at least two, at least three, or more) lumens each extending therethrough, and the lumen(s) is/are lined with an endothelial cell layer; (ii) a second structure defining a second chamber, the second chamber comprising tissue-specific cells therein; and (iii) a membrane located at an interface region between the first chamber and the second chamber to separate the first chamber from the second chamber, the membrane including a first side facing toward the first chamber and a second side facing toward the second chamber; and (b) flowing a first fluid through the lumen(s). In some embodiments, the method can further comprise perfusing the second chamber with a second fluid.

In some embodiments, the method can further comprise detecting a response of blood vessel-associated cells (e.g., endothelial cells and/or pericytes) and/or tissue specific cells in the device and/or detecting at least one component (e.g., a cytokine, molecule, or ion secreted or consumed by the cells in the device) present in an output fluid from the device. Any suitable methods of detecting different types of cell response may be used, including, but not limited to, cell labeling, immunostaining, optical or microscopic imaging (e.g., immunofluorescence microscopy and/or scanning electron microscopy), gene expression analysis, cytokine/chemokine secretion analysis, mass spectrometry analysis, metabolite analysis, polymerase chain reaction, immunoassays, ELISA, gene arrays, and any combinations thereof.

In some embodiments, the methods described herein can further comprise contacting the tissue-specific cells and/or endothelial cell layer with a test agent. Non-limiting examples of the test agents include proteins, peptides, nucleic acids, antigens, nanoparticles, environmental toxins or pollutants, small molecules, drugs or drug candidates, vaccine or vaccine candidates, pro-inflammatory agents, viruses, bacteria, unicellular organisms, cytokines, and any combinations thereof. By detecting the response(s) of the cells treated with the test agent and comparing the responses to response(s) of non-treated cells, an effect of the test agent on the cells can be determined.

The above summary of the embodiments described herein is not intended to represent each embodiment, or every aspect, of the present invention. This is the purpose of the figures and detailed description that follow.

### BRIEF DESCRIPTION OF THE DRAWINGS

FIG. 1 illustrates a block diagram of a system employing an example device in accordance with an embodiment described herein.

FIG. 2A illustrates a perspective view of a device in accordance with an embodiment.

FIG. 2B illustrates an exploded view of the device of FIG. 2A.

FIG. 3A is a schematic diagram showing cross-section of an example device in accordance with an embodiment described herein. The device 200 comprises two compartments, separated by a membrane. The two compartments each independently comprises an extracellular matrix gel 251 and at least one type of cells from a neurovascular unit (e.g., but not limited to pericytes 253, astrocytes 255, and neurons 257).

FIG. 3B is photograph showing top view of the example device 200 of FIG. 3A.

FIG. 3C (scale bar of 200 μm) is a fluorescent immunostaining image showing an example of implementation of the example device. In this embodiment, human cerebral endothelial cells lining the lumen 290 were co-cultured with astrocytes. The endothelial cells were derived from human cortex. They were seeded in the lumen by direct injection into the device in two rounds. In one of the rounds, the device was incubated upside-down until the cells adhered thereto.

FIG. 4 illustrates a system diagram employing at least one device described herein, which can be fluidically connected to another device described herein, an art-recognized organ-on-a-chip device, and/or to fluid sources.

FIG. 5A illustrates a device comprising (i) a first structure defining at least one first chamber; (ii) a second structure defining at least two second chambers; (iii) a membrane located at an interface region between the first stricture and the second structure to separate the first chamber from the two second chambers.

FIG. 5B illustrates a device comprising (i) a first structure defining at least two first chambers; (ii) a second structure defining at least one second chamber; (iii) a membrane located at an interface region between the first structure and the second structure to separate the first two chambers from the second chamber.

FIG. 6A shows a cytokine release profile in 3D devices according to one embodiment described herein normalized to unstimulated devices with an endothelial lumen (n=3-5).

FIG. 6B shows a cytokine release profile in Transwells normalized to unstimulated wells with an endothelial monoculture (n=3). “Endo” refers to an endothelial cell monoculture; “Endo+Astro” refers to an endothelial cell and astrocyte co-culture; and “Endo+Peri” refers to an endothelial cell and pericyte co-culture.

FIG. 7A illustrates a schematic diagram of a polydimethylsiloxane (PDMS) structure used to generate a three-dimensional blood brain-barrier (BBB) chip 700 (left) and an illustration of a cross-section through the chip 700 showing the PDMS channel 702 containing a collagen gel 704 made with viscous fingering and a central lumen (right).

FIG. 7B is a photograph of the 3D BBB chip 700 of FIG. 7A on the stage of an inverted microscope.

FIG. 7C illustrates time-lapse images of a viscous fingering method used to generate a generally cylindrical collagen gel in the 3D BBB chip 700 according to one embodiment showing a microchannel 707 before (t=1) infusion of a neutralized collagen gel containing dispersed human astrocytes (t=2), which was then followed by injection of a low viscosity liquid 706 driven by hydrostatic pressure to initiate “finger” formation in the center of the gel (t=3), and eventually a continuous hollow cylindrical lumen 710 throughout the length of the device (t=4 (bar, 500 μm). The time course from t=1 to t=4 is user dependent but can be accomplished in, e.g., less than about 30 sec.

FIG. 7D is a graph showing the correlation between the hydrostatic pressures used to drive the fingering process and the resulting lumen diameter (* p<0.05 Student's t-test, n=3).

FIG. 7E is a low magnification micrograph of an entire device 708 containing a lumen 710 filled with fluid, formed, e.g., as described in FIG. 7C (dashed lines, delineate the edges of the channel(bar, 3 mm).

FIG. 7F (bar, 100 μm) is a second harmonic generation image of the collagen distribution in the 3D BBB chip 708 of FIG. 7E.

FIG. 7G (bar, 100 μm) is an intensity generated voxel illustration of the FIG. 7F.

FIG. 7H (bar, 50 μm) is a high magnification of the second harmonic generation image of FIG. 7F showing the collagen microstructure in the generally cylindrical gel within the 3D BBB chip 708.

FIG. 8A illustrates a fluorescence confocal micrograph of an engineered brain microvessel viewed from the top showing cell distributions in a 3D BBB chip including brain microvascular endothelium.

FIG. 8B illustrates a low-magnification fluorescence confocal micrograph of a cross-sectional view of the engineered brain microvessel of FIG. 8A.

FIG. 8C illustrates a high-magnification fluorescence confocal micrograph of the rectangular area of the cross-sectional view of the engineered brain of FIG. 8B.

FIG. 8D illustrates a fluorescence confocal micrograph of an engineered brain microvessel viewed from the top showing cell distributions in a 3D BBB chip including endothelium with prior plating of brain pericytes on the surface of the gel in the central lumen.

FIG. 8E illustrates a low-magnification fluorescence confocal micrograph of a cross-sectional view of the engineered brain microvessel of FIG. 8D.

FIG. 8F illustrates a high-magnification fluorescence confocal micrograph of the rectangular area of the cross-sectional view of the engineered brain of FIG. 8E.

FIG. 8G illustrates a fluorescence confocal micrograph of an engineered brain microvessel viewed from the top showing cell distributions in a 3D BBB chip including endothelium with brain astrocytes embedded in the surrounding gel.

FIG. 8H illustrates a low-magnification fluorescence confocal micrograph of a cross-sectional view of the engineered brain microvessel of FIG. 8G.

FIG. 8I illustrates a high-magnification fluorescence confocal micrograph of the rectangular area of the cross-sectional view of the engineered brain of FIG. 8H.

FIG. 8J is a schematic illustration of endothelial cells populating a 3D vessel structure.

FIG. 8K is a schematic illustration of endothelial cells and pericytes populating a 3D vessel structure.

FIG. 8L is a schematic illustration of endothelial cells and astrocytes populating a 3D vessel structure.

FIG. 9A (is a perspective view of a 3D reconstruction of a confocal fluorescence micrograph showing a monolayer of brain microvascular endothelial cells lining the lumen of an engineered vessel in the 3D BBB chip showing F-actin staining 806 and collagen IV staining 812.

FIG. 9B shows a higher magnification view of staining for F-actin (bar, 80 μm).

FIG. 9C shows a higher magnification view of staining for collagen IV (bar, 80 μm).

FIG. 9D (bar, 40 μm) shows a cross-sectional view illustrating the accumulation of a linear pattern of basement membrane collagen IV staining 812 beneath F-actin 806 containing endothelial cells.

FIG. 10A shows fluorescence micrographs of BBB chips containing a generally cylindrical collagen gel viewed from above with a lining endothelial monolayer (left) and an empty collagen lumen (right) after five days of culture. The images were recorded at 0 seconds (top) and about 500 (bottom) seconds after injection of fluorescently-labeled 3 kDa dextran to analyze the dynamics of dextran diffusion and visualize endothelial barrier function in the 3D BBB chip. The presence of the endothelium (left) significantly restricted dye diffusion compared to gels without cells (right).

FIG. 10B illustrates apparent permeabilities of the endothelium cultured in the 3D BBB chip calculated from the diffusion of about 3 kDa dextran with an endothelial monolayer (Endo; n=6), an endothelial monolayer surrounded by astrocytes (Endo+Astro; n=3), and an endothelial monolayer surrounded by pericytes (Endo+Peri; n=3). Error bars indicate S.E.M.; * p<0.05, Student's t-test.

FIG. 11A is a diagrammatic representation of the profile of cytokine release for 5 inflammatory cytokines (i.e., G-CSF, GM-CSF, IL-6, IL-8, IL-17) in 3D BBB chips according to one embodiment.

FIG. 11B is a diagrammatic representations of the profile of cytokine release for 5 inflammatory cytokines (i.e., G-CSF, GM-CSF, IL-6, IL-8, IL-17) in a Transwell.

FIG. 11C illustrates the release of G-CSF, IL-6, and IL-8 in the 3D BBB chips of FIG. 11A under basal conditions and when stimulated with TNF-α, normalized for culture area (* p<0.05, ** p<0.01, *** p<0.001, **** p<0.0001. Multiple-comparison ANOVA with Bonferroni's comparisons test; n=4-7 for 3D BBB chips).

FIG. 11D illustrates the release of G-CSF, IL-6, and IL-8 in the Transwell of FIG. 11B under basal conditions and when stimulated with TNF-α, normalized for culture area (* p<0.05, ** p<0.01, *** p<0.001, **** p<0.0001. Multiple-comparison ANOVA with Bonferroni's comparisons test; n=3 for Transwells).

FIG. 12A illustrates human cerebral cortex microvascular endothelial cells expressing VE-cadherin at an intercellular adherens junction.

FIG. 12B illustrates human cerebral cortex microvascular endothelial cells expressing the tight junction protein ZO-1 at an intercellular adherens junction.

FIG. 12C illustrates human astrocytes displaying differential expression of glial fibril acidic protein (GFAP).

FIG. 12D illustrates human brain-derived pericytes expressing alpha smooth muscle actin (α-SMA) lacking the endothelial markers.

FIG. 12E illustrates human brain-derived pericytes expressing alpha smooth muscle actin (α-SMA) lacking VE-Cadherin.

FIG. 12F illustrates human brain-derived pericytes expressing alpha smooth muscle actin (α-SMA) lacking PECAM.

FIG. 12G illustrates the cells of FIG. 12F being stained with phalloidin, showing that the cells clearly do not form a continuous monolayer.

FIG. 13 illustrates the co-culture of human brain microvascular endothelial cells and pericytes in a 3D BBB chip according to the embodiments described herein. Specifically, FIG. 13 is a perspective view of a brain microvascular endothelium with prior plating of brain pericytes on the surface of the gel in the central lumen.

FIG. 14A shows the apparent permeability values of human brain microvascular endothelial cells, endothelial cells and astrocytes, and endothelial cells and pericytes in static Transwell cultures. Pₐₚₚ values were evaluated using about 5 min assay with about 3 kDa Dextran after about 120 hrs of culture (n=3).

FIGS. 14B show the apparent permeability values of human brain microvascular astrocytes,and pericytes in static Transwell cultures. Pₐₚₚ values were evaluated using about 5 min assay with about 3 kDa Dextran after about 120 hrs of culture (n=3).

FIG. 15 shows TEER values of human brain microvascular endothelial cells, astrocytes, and pericytes in static Transwell cultures. TEER values were recorded after about 120 hrs of culture (n=3).

FIG. 16A shows a comparison of cytokine release profiles after inflammatory stimulation of GM-CSF with TNF-α in a microfluidic 3D BBB chip according to the embodiments described herein versus static Transwell cultures.

FIG. 16B shows a comparison of cytokine release profiles after inflammatory stimulation of IL17 with TNF-α in a microfluidic 3D BBB chip according to the embodiments described herein versus static Transwell cultures.

FIG. 16C show a comparison of cytokine release profiles after inflammatory stimulation of G-CSF with TNF-α in a microfluidic 3D BBB chip according to the embodiments described herein versus static Transwell cultures.

FIG. 16D show a comparison of cytokine release profiles after inflammatory stimulation of IL6 with TNF-α in a microfluidic 3D BBB chip according to the embodiments described herein versus static Transwell cultures.

FIG. 16E show a comparison of cytokine release profiles after inflammatory stimulation of IL8 with TNF-α in a microfluidic 3D BBB chip according to the embodiments described herein versus static Transwell cultures.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

Aspects described herein stem from, at least in part, design of devices that combine creation of a three-dimensional hollow structure in an extracellular matrix protein gel, e.g., by viscous fingering, with compartmentalization of different cell types using one or multiple membranes. Such design can allow for a controlled and physiologically realistic co-culture of endothelialized lumen(s) in one chamber with monolayers and/or three-dimensional cultures of tissue-specific cells in other chambers, where the chambers are aligned (e.g., vertically) with each other with one or more membranes separating them from each other. For example, in some embodiments, the design can allow for realistic co-culture of endothelium, pericytes, astrocytes and neurons in a configuration and in a matrix that is more realistic than what can be achieved with existing Transwell or microfluidic blood-brain barrier models, which only allow for co-culture of flat monolayers. In one aspect, the inventors have used such devices to mimic the organization and/or function of a blood brain barrier in vitro. For example, the inventors have patterned a three-dimensional, endothelial cell-lined lumen or pericyte/endothelial cell-lined lumen, e.g., with circular cross-sectional geometries, through a first permeable matrix (e.g., extracellular matrix gel such as collagen) disposed in a first channel to mimic the structure of blood vessels in vitro, and also have populated a second channel that is separated from the first channel by a membrane, with astrocytes and/or neurons. In particular, in some embodiments, astrocytes can be cultured on one side of the membrane facing the second channel, and neurons can be distributed in a second permeable matrix (e.g., extracellular matrix gel such as a protein mixture gel representing extracellular microenvironment such as MATRIGEL®) that is disposed in the second microchannel. Not only does the first permeable matrix comprise an endothelium-lined lumen or a pericyte/endothelium-lined lumen extending therethrough, in some embodiments, the first permeable matrix can also comprise cells that typically wrap around endothelium of blood vessels in vivo (e.g., pericytes). Accordingly, the inventors, in one aspect, have developed a neurovascular co-culture with an organization that is highly reminiscent of the organization of the neurovascular unit in vivo - endothelial cells facing an open lumen, and interacting with a matrix (e.g., an extracellular matrix) comprising pericytes on their basal side, whereas a layer of astrocytes separates the perivascular gel from a neuronal compartment, in which neurons grow and interact to form a neuronal network. By culturing appropriate cell types in different compartments, the devices can be used to mimic organization and/or function of different tissues. Accordingly, embodiments of various aspects described herein relate to devices for simulating a function of a tissue and methods of making and using the same.

While in some embodiments, the devices described herein are suitable for modeling a blood-brain barrier, the devices described herein can also be used for other organs-on-a-chip requiring at least a three-dimensional endothelialized lumen that interacts with a co-culture of cells in monolayers and/or three-dimensional structures including, but not limited to, Lung-on-a-Chip, Skin-on-a-Chip, Liver-on-a-Chip, Gut-on-a-Chip, Heart-on-a-Chip, Eye-on-a-Chip, Kidney-on-a-Chip, and others. Accordingly, in some embodiments, the devices described herein can be used to model diseases other than brain diseases such as, but not limited to, respiratory diseases, skin diseases, liver diseases, gastrointestinal diseases, heart diseases, and ocular diseases.

Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used throughout the drawings and the following description to refer to the same or like items. It is understood that the phrase “an embodiment” encompasses more than one embodiment and is, thus, not limited to only one embodiment for brevity's sake.

### Example Devices for Simulating a Function of a Tissue

Some aspects described herein relate to devices for simulating a function of a tissue. The devices generally comprise (i) a first structure defining a first chamber, the first chamber comprising a first permeable matrix disposed therein, wherein the first permeable matrix comprises at least one or a plurality of (e.g., at least two, at least three or more) lumens each extending therethrough; (ii) a second structure defining a second chamber, the second chamber comprising cells disposed therein; and (iii) a membrane located at an interface region between the first chamber and the second chamber to separate the first chamber from the second chamber, the membrane including a first side facing toward the first chamber and a second side facing toward the second chamber. The cells disposed in the second chamber can be adhered on the second side of the membrane and/or distributed in a second permeable matrix disposed in the second chamber.

Thus, in one aspect described herein, a device for simulating a function of a tissue comprises (i) a first structure defining a first chamber, the first chamber comprising a first permeable matrix disposed therein, wherein the first permeable matrix comprises at least one or a plurality of (e.g., at least two, at least three or more) lumens each extending therethrough; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the second side comprises cells of a first type adhered thereon.

In some embodiments, the cells of the first type adhering on the second side of the membrane can form a cell monolayer and/or a three-dimensional or stratified structure.

In some embodiments, the second side of the membrane can comprise a permeable matrix layer on which the cells of the first type adhered.

In some embodiments, second chamber can comprise a second permeable matrix disposed therein. In some embodiments, the second permeable matrix can comprise cells of a second type. In some embodiments, the second permeable matrix can comprise at least one or more lumens each extending therethrough. In some embodiments, the lumen(s) in the second permeable matrix can comprise cells.

Another aspect described herein is a device for simulating a function of a tissue, comprising: (i) a first structure defining a first chamber, the first chamber comprising a first permeable matrix disposed therein, wherein the first permeable matrix comprises at least one or a plurality of (e.g., at least two, at least three or more) lumens each extending therethrough; (ii) a second structure defining a second chamber, the second chamber comprising a second permeable matrix disposed therein; and (iii) a membrane located at an interface region between the first chamber and the second chamber to separate the first chamber from the second chamber, the membrane including a first side facing toward the first chamber and a second side facing toward the second chamber.

In some embodiments, the second side of the membrane can comprise cells of a first type adhered thereon.

In some embodiments of this aspect and other aspects described herein, the lumen(s) can be configured to mimic a duct or sinus of a tissue or an organ or to mimic a blood vessel. For example, in some embodiments, the lumen(s) can be lined with at least one layer of cells comprising blood vessel-associated cells and/or tissue-specific cells (e.g., tissue-specific epithelial cells). Examples of blood vessels-associated cells include, but are not limited to, endothelial cells, fibroblasts, smooth muscle cells, pericytes, and a combination of two or more thereof. In one embodiment, the lumen(s) can be lined with an endothelial cell monolayer. In one embodiment, the lumen(s) can be lined with pericytes (e.g., a sparse layer of pericytes) covered by an endothelial cell monolayer.

As used herein, the term “monolayer” refers to a single layer of cells on a growth surface, on which no more than 10% (e.g., 10%, 9%, 8%, 7%, 6%, 5%, 4%, 3%, 2%, 1%, or 0%) of the cells are growing on top of one another, and at least about 90% or more (e.g., at least about 95%, at least 98%, at least 99%, and up to 100%) of the cells are growing on the same growth surface. In some embodiments, all of the cells are growing side-by side, and can be touching each other on the same growth surface. The condition of the cell monolayer can be assessed by any methods known in the art, e.g., microscopy, and/or immunostaining for cell-cell adhesion markers. In some embodiments where the cell monolayer comprises an endothelial cell monolayer, the condition of the endothelial cell monolayer can be assessed by staining for any art-recognized cell-cell adhesion markers in endothelial cells including, but not limited to, VE-cadherin.

In some embodiments, the second permeable matrix can comprise at least one or more lumens each extending therethrough. In some embodiments, the lumen(s) in the second permeable matrix can comprise cells.

In some embodiments of this aspect and other aspects described herein, the second permeable matrix can comprises cells of a second type distributed therein.

In some embodiments of this aspect and other aspects described herein, the first permeable matrix can comprise cells of a third type distributed therein.

In some embodiments of this aspect and other aspects described herein, the first side of the membrane can comprise cells of a fourth type adhered thereon.

In some embodiments, the cells of the first type, second type, third type, and/or fourth type can each independently comprise a type of tissue-specific cell. Appropriate tissue-specific cells can be selected depending on the organization and/or function of a tissue to be modeled. For example, tissue-specific cells may be parenchymal cells (e.g., epithelial cells) derived from a tissue or an organ including, but not limited to, a lung, a liver, a kidney, a skin, an eye, a brain, a blood-brain-barrier, a heart, a gastrointestinal tract, airways, a reproductive organ, a combination of two or more thereof, or the like.

In some embodiments of various aspects described herein, the second side of the membrane can comprise blood vessel-associated cells, including, e.g., but not limited to endothelial cells and/or pericytes. In one embodiment, the second side of the membrane can comprise an endothelial cell monolayer. In one embodiment, the second side of the membrane can comprise a layer comprising pericytes and an endothelial cell monolayer, wherein the endothelial cell monolayer covers the pericyte-comprising layer. In these embodiments where the second side comprises blood vessel-associated cells, the lumen(s) can be lined with tissue-specific cells (e.g., ductal epithelial cells) to simulate a function of a duct or sinus of a tissue or an organ. In some embodiments, the first permeable matrix can comprise connective tissue cells embedded therein.

In some embodiments, the tissue specific cells cultured in the devices described herein can comprise cells that are present in a cerebral endothelial microenvironment to mimic the organization, function, and/or physiology of a blood-brain-barrier. Accordingly, some further aspects described herein relates to devices for simulating a function of a blood-brain-barrier. In one aspect, a device for simulating a function of a blood-brain-barrier comprises: (i) a first structure defining a first chamber, the first chamber comprising a first permeable matrix disposed therein, wherein the first permeable matrix comprises at least one or a plurality of (i.e., at least two or more, including, e.g., at least three or more) lumens each extending therethrough, and the lumen(s) is/are lined with at least one endothelial cell layer; (ii) a second structure defining a second chamber, the second chamber comprising a first type of brain microenvironment-associated cells distributed therein; and (iii) a membrane located at an interface region between the first chamber and the second chamber to separate the first chamber from the second chamber, the membrane comprising a first side facing toward the first chamber and a second side facing toward the second chamber.

In some embodiments, the first type of brain microenvironment-associated cells can be adhered on the second side of the membrane facing the second chamber. In some embodiments, the first type of brain microenvironment-associated cells can be embedded in a second permeable matrix disposed in the second chamber. Examples of the first type of brain microenvironment-associated cells include, but are not limited to, astrocytes, microglia, neurons, and a combination of two or more thereof.

In some embodiments, the first permeable matrix can comprise a second type of brain microenvironment-associated cells distributed therein. Examples of the second type of brain microenvironment-associated cells include, but are not limited to, pericytes, astrocytes, microglia, fibroblasts, smooth muscle cells, or a combination of two or more thereof.

In some embodiments, the lumen(s) can be lined with pericytes (e.g., a sparse layer of pericytes) covered by an endothelial cell monolayer.

In some embodiments, the device can comprise: (i) a first structure defining a first chamber, the first chamber comprising a first permeable matrix disposed therein, wherein the first permeable matrix comprises astrocytes embedded therein and at least one or a plurality of (e.g., at least two, at least three or more) lumens each extending therethrough; and wherein the lumen(s) is/are lined with a cell layer comprising pericytes and an endothelial cell monolayer covering the pericyte-comprising layer; (ii) a second structure defining a second chamber; and (iii) a membrane located at an interface region between the first chamber and the second chamber to separate the first chamber from the second chamber, the membrane comprising a first side facing toward the first chamber and a second side facing toward the second chamber.

In some embodiments, the device can comprise: (i) a first structure defining a first chamber, the first chamber comprising a first permeable matrix disposed therein, wherein the first permeable matrix comprises at least one or a plurality of (e.g., at least two, at least three or more) lumens each extending therethrough, and the lumen(s) is/are lined with a cell layer comprising pericytes and an endothelial cell monolayer covering the pericyte-comprising layer; (ii) a second structure defining a second chamber, the second chamber comprising a second permeable matrix disposed therein, the second permeable matrix comprising brain microenvironment-associated cells (including, e.g., but not limited to neurons) distributed therein; and (iii) a membrane located at an interface region between the first chamber and the second chamber to separate the first chamber from the second chamber, the membrane comprising a first side facing toward the first chamber and a second side facing toward the second chamber, wherein the second side can comprise brain microenvironment-associated cells (including, but not limited to, astrocytes, microglia, neurons, and any combinations thereof) adhered thereon. In one embodiment, the second side can comprise astrocytes adhered thereon. In one embodiment, the first permeable matrix can comprise pericytes.

In another aspect, a device for simulating a function of a blood-brain-barrier comprises: (i) a first structure defining a first chamber, the first chamber comprising a first permeable matrix disposed therein, wherein the first permeable matrix comprises at least one or a plurality of (i.e., at least two or more, including, e.g., at least three or more) lumens each extending therethrough, and the lumen(s) is/are lined with at least one layer of cells mimicking a brain sinus; (ii) a second structure defining a second chamber, the second chamber comprising blood vessel-associated cells (e.g., endothelial cells and/or pericytes) distributed therein; and (iii) a membrane located at an interface region between the first chamber and the second chamber to separate the first chamber from the second chamber, the membrane comprising a first side facing toward the first chamber and a second side facing toward the second chamber. In some embodiments, the blood vessel-associated cells can be adhered on the second side of the membrane facing the second chamber.

It is commonly believed that the native brain endothelial cells are usually exposed to a high shear stress. Accordingly, in some embodiments, application of a mechanical strain/stress to the brain cells can be used in place of a high-shear flow.

Use of the devices described herein to model a blood-brain barrier are provided herein as illustrative examples and are not intended to be in any way limiting. Those of skill in the art will realize that the devices described herein can be adapted to mimic function of any portion of a tissue or organ in any living organisms, e.g., vertebrates (e.g., but not limited to, human subjects or animals such as fish, birds, reptiles, and amphibians), invertebrates (e.g., but not limited to, protozoa, annelids, mollusks, crustaceans, arachnids, echinoderms and insects), plants, fungi (e.g., but not limited to mushrooms, mold, and yeast), and microorganisms (e.g., but not limited to bacteria and viruses) in view of the specification and examples provided herein. Further, a skilled artisan can adapt methods of uses described herein for various applications of different tissue-mimic devices.

Methods of creating three-dimensional lumen structures in permeable matrices are known in the art. For example, a method as described in Bischel et al. J Lab Autom. (2012) 17: 96-103; and Bischel et al. Biomaterials (2013) 34: 1471-1477) can be used to create at least one three-dimensional lumen in the first permeable matrix disposed in the first chamber. The Bischel method generally relies on a phenomenon called “viscous fingering,” which was used to create lumens with a circular cross-section in microfluidic channels after those channels have been filled with a highly viscous solution of matrix proteins. The method relies on a pressure driven flow of a fluid with low viscosity through the high viscosity matrix phase; instead of washing away all high-viscosity liquid, the low-viscosity liquid “fingers” through, thus creating a circular lumen in the surrounding matrix. However, the Bischel reference does not teach or suggest, e.g., creating a lumen in a permeable matrix disposed on one side of a porous membrane, while the other side can comprise cells adhered on the membrane and/or a separate permeable matrix disposed thereon, wherein the separate permeable matrix can optionally comprise cells distributed therein.

In some embodiments of this aspect and other aspects described herein, the lumen(s) can be formed by a process comprising (i) providing the first chamber filled with a viscous solution of the first matrix molecules; (ii) flowing at least one pressure-driven fluid with a viscosity lower than that of the viscous solution through the viscous solution to create one or more lumens each extending through the viscous solution; and (iii) gelling, polymerizing, and/or crosslinking the viscous solution. Thus, one or more lumens each extending through the first permeable matrix can be created.

The solution of the first matrix molecules can have a viscosity that is high enough to form a defined structure but also allows a fluid of a lower viscosity to disperse through the viscous solution, e.g., via surface tension-based passive pumping and/or pressure-driven flow, and to remove the portion of the viscous solution, thereby creating one or more lumens within the viscous solution, after which polymerization of the remaining viscous solution results in a matrix gel comprising one or more lumens each extending therethrough. In some embodiments, the solution of the first matrix molecules can have a viscosity of about 2 cP to about 40 cP.

The fluid of a lower viscosity that is dispersed through the viscous solution of the first matrix molecules can vary with the viscosity of the viscous solution. In general, the more viscous the first matrix molecule solution is, the higher the viscosity of the fluid may be required to push through the viscous solution and to create lumen(s) therein. In some embodiments, the fluid used to disperse through the viscous solution can have a viscosity of about 0.5 cP to about 5 cP.

The pressure (and/or flow rate) used to disperse the fluid through the viscous solution of the first matrix molecules can range from about 0.5 cm H₂O to about 20 cm H₂O.

After creating the lumen(s) each extending through the viscous solution of the first matrix molecules, the viscous solution is then subjected to a polymerization condition, which can vary with different matrix material properties. For example, when the first matrix molecule solution comprises collagen I, a gel can be formed when the solution is incubated at about 37° C. A skilled person in the art can determine appropriate polymerization condition based on the selected matrix material(s) and/or cell compatibility (if the solution comprises cells).

Other suitable methods can be used to create at least one or more three-dimensional lumen structures in a permeable matrix. As another example, at least one or more three-dimensional lumens can be created in a permeable matrix by introducing an extractable object (e.g., a microneedle, a thin needle, a suture, a thread and/or any other moldable placeholders) into a chamber as a rigid placeholder. After formation of a permeable matrix surrounding the extractable object, the extractable object (e.g., a microneedle, a thread) can be removed, e.g., by using a physical force (e.g., pulling out a microneedle or thread) and/or dissolving the extractable object with temperature changes and/or exposure to light. Alternatively, a stimuli-responsive material can be used to form a permeable matrix in the chamber and then one or more lumens can be formed by directing a stimulus to a portion of the matrix where lumen(s) are desired to be created. For example, a focused light (e.g., a laser light in mono or two photo configuration) can be shone through a light-sensitive matrix such that the matrix material that is exposed to the light is degraded, thus creating lumen(s) in the matrix. In some embodiments, lumens can be formed by localized photopolymerization.

As used herein, the term “lumen” refers to a passageway, conduit, or cavity formed within a matrix gel. The lumen(s) can have a cross-section of any shape, including, e.g., but not limited to circular, elliptical, square, rectangular, triangular, semi-circular, irregular, free-form and any combinations thereof In some embodiments, the lumen(s) can have a circular cross-section. The lumen(s) can form a substantially linear and/or non-linear passageway or conduit within a matrix gel. Thus, the lumen(s) is/are not limited to straight or linear passageways or conduits and can comprise curved, angled, or otherwise non-linear passageway or conduit. It is to be further understood that a first portion of a lumen can be straight, and a second portion of the same lumen can be curved, angled, or otherwise non-linear. In some embodiments, the lumen(s) can be branched, e.g., a portion of a main lumen can be extended to form at least two or more (e.g., two, three, four, or more) passageways or conduits diverging from the main lumen.

The dimensions of the lumen(s) can vary with a number of factors, including, but not limited to dimensions of the channels, relative viscosities between a viscous solution of first matrix molecules and a fluid flowing through the viscous solution, volumetric flow rate and/or pressure of the fluid flowing through the viscous solution, and any combination thereof In some embodiments, the lumen(s) can have a dimension of about 10 μm to about 800 μm. In some embodiments, the lumen(s) can have a dimensions less than 10 μm, including, e.g., less than 9 μm, less than 8 μm, less than 7 μm, less than 6 μm, or lower.

In accordance with embodiments of various aspects described herein, the first chamber comprises a first permeable matrix disposed therein. In some embodiments, the second chamber can comprise a second permeable matrix. The term “permeable matrix” or “permeable matrices” as used herein means a matrix or scaffold material that permits passage of a fluid (e.g., liquid or gas), a molecule, a whole living cell and/or at least a portion of a whole living cell, e.g., for formation of cell-cell contacts. In some embodiments, permeable matrices also encompass selectively permeable matrices. The term “selectively permeable matrix” as used herein refers to a matrix material that permits passage of one or more target group or species, but act as a barrier to non-target groups or species. For example, a selectively-permeable matrix can allow transport of a fluid (e.g., liquid and/or gas), nutrients, wastes, cytokines, and/or chemokines through the matrix, but does not allow whole living cells to migrate therethrough. In some embodiments, a selectively-permeable matrix can allow certain cell types to migrate therethrough but not other cell types. In some embodiments, the permeable matrices can swell upon contact with a liquid (e.g., water and/or culture medium). For example, the permeable matrices can be gels or hydrogels. In some embodiments, the permeable matrices can be a non-swollen polymer upon contact with a liquid (e.g., water and/or culture medium). In some embodiments, the permeable matrices can form a mesh and/or porous network.

The lumen(s) described herein can be defined in a permeable polymer matrix. Any method described herein or any suitable method may be used, including, but not limited to inserting an elongated structure (e.g., a cylindrical, elongated structure such as a microneedle) into the polymer matrix solution. See, e.g., Park et al., Biotechnol. Bioeng. (2010) 106(1): 138-148 for additional information about creating microporous matrix for cell/tissue culture models, the content of which is incorporated herein by reference. Non-limiting examples of methods that can be used to create permeable matrices with or without a lumen therein are also described, e.g., in Annabi et al., Tissue Eng Part B Rev. (2010) 16: 371-383, the content of which is incorporated herein by reference. The methods described in the cited references can be applied to fabrication of polymer matrices other than hydrogels.

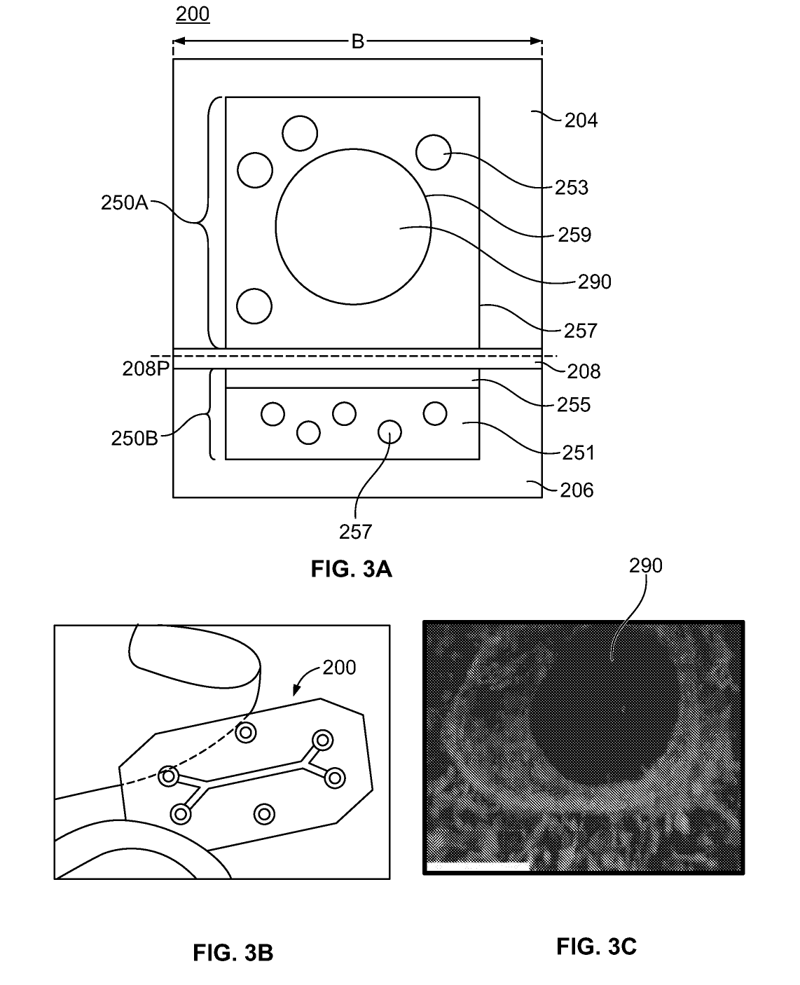

In accordance with various aspects described herein, the first structure defines a first chamber, and the second structure defines a second chamber. While the first chamber and the second chamber can be in any geometry or three-dimensional structure, in some embodiments, the first chamber and the second chamber can be configured to be form channels. FIG. 2A illustrates a perspective view of the device in accordance with an embodiment. As shown in FIG. 2A, the device 200 (also referred to reference numeral 102) can include a body 202 comprising a first structure 204 and a second structure 206 in accordance with an embodiment. The body 202 can be made of an elastomeric material, although the body can be alternatively made of a non-elastomeric material, or a combination of elastomeric and non-elastomeric materials. It should be noted that the microchannel design 203 is only exemplary and not limited to the configuration shown in FIG. 2A. While operating chambers 252 (e.g., as a pneumatics means to actuate the membrane 208, see the International Appl. No. PCT/US2009/050830 for further details of the operating chambers, the content of which is incorporated herein by reference in its entirety) are shown in FIGS. 2A-2B, they are not required in all of the embodiments described herein. In some embodiments, the devices do not comprise operating chambers on either side of the first chamber and the second chamber. For example, FIG. 3A shows a device that does not have an operating channel on either side of the first chamber and the second chamber. In other embodiments, the devices described herein can be configured to provide other means to actuate the membrane, e.g., as described in the International Pat. Appl. No. PCT/US2014/071570, the content of which is incorporated herein by reference in its entirety.

In some embodiments, various organ chip devices described in the International Patent Application Nos. PCT/US2009/050830, PCT/US2012/026934, PCT/US2012/068725, PCT/US2012/068766, PCT/US2014/071611, and PCT/US2014/071570, the contents of each of which are incorporated herein by reference in their entireties, can be used or modified to form the devices described herein. For example, the organ chip devices described in those patent applications can be modified to have at least one of the chambers comprising a first permeable matrix disposed therein, wherein the first permeable matrix comprises at least one or a plurality of (e.g., at least two, at least three or more) lumens each extending therethrough, and to have another chamber comprising cells cultured therein, e.g., on the membrane and/or in a second permeable matrix optionally disposed in the second chamber.

The device in FIG. 2A can comprise a plurality of access ports 205. In addition, the branched configuration 203 can comprise a tissue-tissue interface simulation region (membrane 208 in FIG. 2B) where cell behavior and/or passage of gases, chemicals, molecules, particulates and cells are monitored. FIG. 2B illustrates an exploded view of the device in accordance with an embodiment. In one embodiment, the body 202 of the device 200 comprises a first outer body portion (first structure) 204, a second outer body portion (second structure) 206, and an intermediary membrane 208 configured to be mounted between the first and second outer body portions 204, 206 when the portions 204, 206 are mounted to one another to form the overall body.

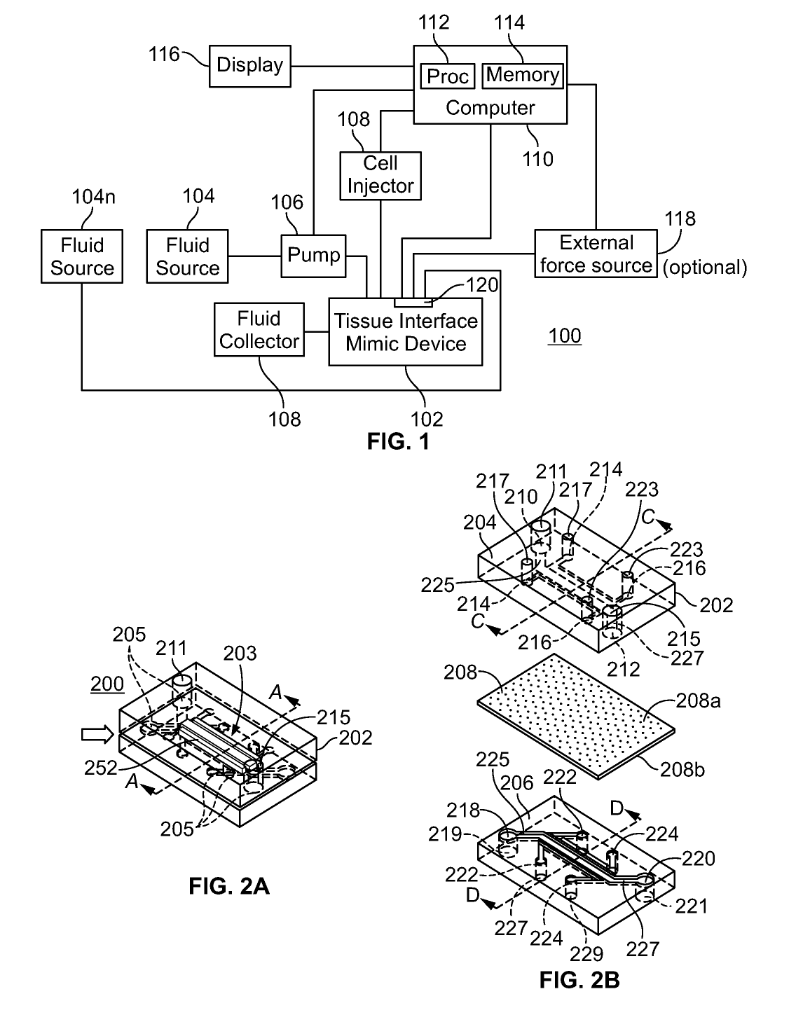

FIG. 2B illustrates an exploded view of the device 200 of FIG. 2A in accordance with an embodiment. As shown in FIG. 2B, the first outer body portion or first structure 204 includes one or more inlet fluid ports 210 in communication with one or more corresponding inlet apertures 211 located on an outer surface of the first structure 204. The device 200 can be connected to the fluid source 104 (see FIG. 1) via the inlet aperture 211 in which fluid travels from the fluid source 104 into the device 200 through the inlet fluid port 210.

Additionally, the first outer body portion or first structure 204 can include one or more outlet fluid ports 212 in communication with one or more corresponding outlet apertures 215 on the outer surface of the first structure 204. In some embodiments, a fluid passing through the device 100 can exit the device 100 to a fluid collector 108 or other appropriate component via the corresponding outlet aperture 215. It should be noted that the device 200 can be set up such that the fluid port 210 is an outlet and fluid port 212 is an inlet.

In some embodiments, as shown in FIG. 2B, the device 200 can comprise an inlet channel 225 connecting the inlet fluid port 210 to a first chamber 250A (see FIG. 3A). The inlet channels 225 and inlet fluid ports 210 can be used to introduce cells, agents (e.g., stimulants, drug candidate, particulates), air flow, and/or cell culture media into the first chamber 250A.

The device 200 can also comprise an outlet channel 227 connecting the outlet fluid port 212 to the first chamber 250A. The outlet channels 227 and outlet fluid ports 212 can also be used to introduce cells, agents (e.g., stimulants, drug candidate, particulates), air flow, and/or cell culture media into the first chamber 250A.

In some embodiments, the first structure 204 can include one or more pressure inlet ports 214 and one or more pressure outlet ports 216 in which the inlet ports 214 are in communication with corresponding apertures 217 located on the outer surface of the device 200. Although the inlet and outlet apertures are shown on the top surface of the first structure 204, one or more of the apertures can alternatively be located on one or more lateral sides of the first structure and/or second structure. In operation, one or more pressure tubes (not shown) connected to the external force source (e.g., pressure source) 118 (FIG. 1) can provide positive or negative pressure to the device via the apertures 217. Additionally, pressure tubes (not shown) can be connected to the device 200 to remove the pressurized fluid from the outlet port 216 via apertures 223. It should be noted that the device 200 can be set up such that the pressure port 214 is an outlet and pressure port 216 is an inlet. It should be noted that although the pressure apertures 217, 223 are shown on the top surface of the first structure 204, one or more of the pressure apertures 217, 223 can be located on one or more side surfaces of the first structure 204.

Referring to FIG. 2B, in some embodiments, the second structure 206 can include one or more inlet fluid ports 218 and one or more outlet fluid ports 220. As shown in FIG. 2B, the inlet fluid port 218 is in communication with aperture 219 and outlet fluid port 220 is in communication with aperture 221, whereby the apertures 219 and 221 are located on the outer surface of the second structure 206. Although the inlet and outlet apertures are shown on the surface of the second structure, one or more of the apertures can be alternatively located on one or more lateral sides of the second structure.

In some embodiments, the second outer body portion and/or second structure 206 can include one or more pressure inlet ports 222 and one or more pressure outlet ports 224. In some embodiments, the pressure inlet ports 222 can be in communication with apertures 227 and pressure outlet ports 224 are in communication with apertures 229, whereby apertures 227 and 229 are located on the outer surface of the second structure 206. Although the inlet and outlet apertures are shown on the bottom surface of the second structure 206, one or more of the apertures can be alternatively located on one or more lateral sides of the second structure. Pressure tubes connected to the external force source (e.g., pressure source) 118 (FIG. 1) can be engaged with ports 222 and 224 via corresponding apertures 227 and 229. It should be noted that the device 200 can be set up such that the pressure port 222 is an outlet and the fluid port 224 is an inlet.

The first chamber 204 and the second chamber 206 can each have a range of width dimension (shown as B in FIG. 3A) between about 200 microns and about 10 mm, or between about 200 microns and about 1,500 microns, or between about 400 microns and about 1,000 microns, or between about 50 and about 2,000 microns. In some embodiments, the first chamber 204 and the second chamber 206 can each have a width of about 500 microns to about 2 mm. In some embodiments, the first chamber 204 and the second chamber 206 can each have a width of about 1 mm.

In some embodiments where the second structure 206 defines at least two or more second chambers 250B, e.g., as shown in FIG. 5A, the width of the second chambers 250B can be smaller than the width of the first chamber 250A. In these embodiments, the first chamber 250A can comprise a permeable matrix disposed therein, wherein the first permeable matrix can comprise more than one lumens 290 extending therethrough. Each lumen 290 can be arranged side-by-side in the first permeable matrix such that it is aligned with a respective second chamber 250B, e.g., as shown in FIG. 5A. In FIG. 5A, the first permeable matrix can comprise one lumen shared by the two second chambers (not shown), or can comprise two lumens each aligned with the corresponding second chamber (as shown).

In some embodiments where the first structure 204 defines at least two or more first chambers 250A, e.g., as shown in FIG. 5B, the width of each of the first chambers 250A can be smaller than the width of the second chamber 250B. In these embodiments, each of the first chambers 250A can comprise a first permeable matrix disposed therein, and the first permeable matrix in each chamber can comprise a lumen 290 extending therethrough. In FIG. 5B, the first permeable matrix in each of the first chambers can comprise a lumen.

In some embodiments, the first structure and/or second structure of the devices described herein can be further adapted to provide mechanical modulation of the membrane. Mechanical modulation of the membrane can include any movement of the membrane that is parallel to and/or perpendicular to the force/pressure applied to the membrane, including, but are not limited to, stretching, bending, compressing, vibrating, contracting, waving, or any combinations thereof. Different designs and/or approaches to provide mechanical modulation of the membrane between two chambers have been described, e.g., in the International Patent App. Nos. PCT/US2009/050830, and PCT/US2014/071570, the contents of which are incorporated herein by reference in their entireties, and can be adapted herein to modulate the membrane in the devices described herein.

In some embodiments, the devices described herein can be placed in or secured to a cartridge. In accordance with some embodiments of some aspects described herein, the device can be integrated into a cartridge and form a monolithic part. Some examples of a cartridge are described in the International Patent App. No. PCT/US2014/047694, the content of which is incorporated herein by reference in its entirety. The cartridge can be placed into and removed from a cartridge holder that can establish fluidic connections upon or after placement and optionally seal the fluidic connections upon removal. In some embodiments, the cartridge can be incorporated or integrated with at least one sensor, which can be placed in direct or indirect contact with a fluid flowing through a specific portion of the cartridge during operation. In some embodiments, the cartridge can be incorporated or integrated with at least one electric or electronic circuit, for example, in the form of a printed circuit board or flexible circuit. In accordance with some embodiments of some aspects described herein, the cartridge can comprise a gasketing embossment to provide fluidic routing.

In some embodiments, the device described herein can be connected to the cartridge by an interconnect adapter that connects some or all of the inlet and outlet ports of the device to microfluidic channels or ports on the cartridge. Some examples of interconnect adapters are disclosed in U.S. Provisional Application No. 61/839,702, filed on Jun. 26, 2013, and the International Patent Application No. PCT/US2014/044417, filed Jun. 26, 2014, the contents of each of which are hereby incorporated by reference in their entirety. The interconnect adapter can include one or more nozzles having fluidic channels that can be received by ports of the device described herein. The interconnect adapter can also include nozzles having fluidic channels that can be received by ports of the cartridge.

In some embodiments, the interconnect adaptor can comprise a septum interconnector that can permit the ports of the device to establish transient fluidic connection during operation, and provide a sealing of the fluidic connections when not in use, thus minimizing contamination of the cells and the device. Some examples of a septum interconnector are described in U.S. Provisional Application No. 61/810,944, filed Apr. 11, 2013, the content of which is incorporated herein by reference in its entirety.

The membrane 208 is oriented along a plane 208P parallel to the x-y plane between the first chamber 250A and the second chamber 250B, as shown in FIG. 3A. It should be noted that although one membrane 208 is shown in FIG. 3A, more than one membrane 208 can be included, e.g., in devices that comprise more than two chambers.

In some embodiments, a membrane can comprise an elastomeric portion fabricated from a styrenic block copolymer-comprising composition, e.g., as described in the International Pat. App. No. PCT/US2014/071611 (the contents of each of which are incorporated herein by reference in its entirety), can be adopted in the devices described herein. In some embodiments, the styrenic block copolymer-comprising composition can comprise styrene-ethylene-butylene-styrene (SEBS), polypropylene, or a combination thereof.

In some embodiments, a porous membrane can be a solid biocompatible material or polymer that is inherently permeable to at least one matter/species (e.g., gas molecules) and/or permits formation of cell-cell contacts. In some embodiments, through-holes or apertures can be introduced into the solid biocompatible material or polymer, e.g., to enhance fluid/molecule transport and/or cell migration. In one embodiment, through-holes or apertures can be cut or etched through the solid biocompatible material such that the through-holes or apertures extend vertically and/or laterally between the two surfaces of the membrane 208A and 208B. It should also be noted that the pores can additionally or alternatively incorporate slits or other shaped apertures along at least a portion of the membrane 208 which allow cells, particulates, chemicals and/or fluids to pass through the membrane 208 from one section of the central channel to the other.

As used herein, the term “co-culture” refers to two or more different cell types being cultured in some embodiments of the devices described herein. The different cell types can be cultured in the same chamber (e.g., first chamber or second chamber) and/or in different chambers (e.g., one cell type in a first chamber and another cell type in a second chamber). For example, the devices described herein can be used to have endothelial cells facing an open lumen in the first chamber, and interacting with the first permeable matrix comprising tissue-specific cells described herein. In some embodiments, the devices described herein comprise at least one or more (including, e.g., at least two or more) endothelium-lined or pericyte/endothelium-lined lumen(s) in the first chamber and tissue specific cells in the second chamber. The tissue specific cells can be adhered on the side of the membrane facing the second chamber and/or distributed in the second permeable matrix disposed in the second chamber.

While embodiments of various aspects described herein illustrate devices comprising at least one or more lumens in the first permeable matrix and/or second permeable matrix to mimic a duct, a sinus, and/or a blood vessel, one can modify the devices described herein to remove the lumen(s) in the first permeable matrix and to leverage the structural shape (e.g., a channel) of the first chamber and/or the second chamber to provide a hollow lumen. In these embodiments, the first chamber and/or the second chamber (e.g., in a form of channels) can be coated with a permeable matrix layer (i.e., of a finite thickness), and then lined with at least one layer of cells. In some embodiments, the permeable matrix layer can be lined with an endothelial cell monolayer. In some embodiments, the permeable matrix layer can be lined with a cell layer comprising pericytes and an endothelial cell monolayer covering the pericyte-comprising layer.

Examples of endothelial cells that can be grown on the inner surface of the lumen(s) in the first chamber include, but are not limited to, cerebral endothelial cells, blood vessel and lymphatic vascular endothelial fenestrated cells, blood vessel and lymphatic vascular endothelial continuous cells, blood vessel and lymphatic vascular endothelial splenic cells, corneal endothelial cells, and any combinations thereof.

The endothelium is the thin layer of cells that line the interior surface of blood vessels and lymphatic vessels, forming an interface between circulating blood or lymph in the lumen(s) and the rest of the vessel wall. Endothelial cells in direct contact with blood are vascular endothelial cells, whereas those in direct contact with lymph are known as lymphatic endothelial cells. Endothelial cells line the entire circulatory system, from the heart to the smallest capillary. These cells reduce turbulence of the flow of blood allowing the fluid to be pumped farther.

The foundational model of anatomy makes a distinction between endothelial cells and epithelial cells on the basis of which tissues they develop from and states that the presence of vimentin rather than keratin filaments separate these from epithelial cells. Endothelium of the interior surfaces of the heart chambers are called endocardium. Both blood and lymphatic capillaries are composed of a single layer of endothelial cells called a monolayer. Endothelial cells are involved in many aspects of vascular biology, including: vasoconstriction and vasodilation, and hence the control of blood pressure; blood clotting (thrombosis & fibrinolysis); atherosclerosis; formation of new blood vessels (angiogenesis); inflammation and barrier function - the endothelium acts as a selective barrier between the vessel lumen and surrounding tissue, controlling the passage of materials and the transit of white blood cells into and out of the bloodstream. Excessive or prolonged increases in permeability of the endothelial monolayer, as in cases of chronic inflammation, can lead to tissue edema/swelling. In some organs, there are highly differentiated endothelial cells to perform specialized ‘filtering’ functions. Examples of such unique endothelial structures include the renal glomerulus and the blood-brain barrier.

Using the devices described herein, one can study biotransformation, absorption, clearance, metabolism, and activation of xenobiotics, as well as drug delivery. The bioavailability and transport of chemical and biological agents across epithelial layers as in the intestine, endothelial layers as in blood vessels, and across the blood-brain barrier can also be studied. The acute basal toxicity, acute local toxicity or acute organ-specific toxicity, teratogenicity, genotoxicity, carcinogenicity, and mutagenicity, of chemical agents can also be studied. Effects of infectious biological agents, biological weapons, harmful chemical agents and chemical weapons can also be detected and studied. Infectious diseases and the efficacy of chemical and biological agents to treat these diseases, as well as optimal dosage ranges for these agents, can be studied. The response of organs in vivo to chemical and biological agents, and the pharmacokinetics and pharmacodynamics of these agents can be detected and studied using the devices described herein. The impact of genetic content on response to the agents can be studied. The amount of protein and gene expression in response to chemical or biological agents can be determined. Changes in metabolism in response to chemical or biological agents can be studied as well using devices described herein.

### Exemplary Methods of Making the Devices Described Herein

In one aspect, a method of making a device for simulating a function of a tissue is described herein. The method comprises: (a) providing a body comprising: (i) a first structure defining a first chamber, at least a portion of the first chamber filled with a viscous solution of first matrix molecules disposed therein, (ii) a second structure defining a second chamber; and (iii) a membrane located at an interface region between the first chamber and the second chamber to separate the first chamber from the second chamber, the membrane including a first side facing toward the first chamber and a second side facing toward the second chamber; (b) flowing at least one pressure-driven fluid with viscosity lower than that of the viscous solution through the viscous solution in the first chamber to create one or more lumens each extending through the viscous solution; (c) gelling, polymerizing, and/or crosslinking the viscous solution in the first chamber, thereby forming a first permeable matrix comprising one or more lumens each extending therethrough; and (d) populating at least a portion of the second chamber with tissue-specific and/or blood vessel-associated cells.

Embodiments of various devices comprising a first chamber, a second chamber, and a membrane can assist in leveraging the control of microfluidic technology for device fabrication. In some embodiments, the devices described herein can be manufactured using any conventional fabrication methods, including, e.g., injection molding, embossing, etching, casting, machining, stamping, lamination, photolithography, or any combinations thereof. Soft lithography techniques are described in “Soft Lithography in Biology and Biochemistry,” by Whitesides, et al., published Annual Review, Biomed Engineering, 3.335-3.373 (2001), as well as “An Ultra-Thin PDMS Membrane As A Bio/Micro-Nano Interface: Fabrication And Characterization”, by Thangawng et al., Biomed Microdevices, vol. 9, num. 4, 2007, p. 587-95, both of which are hereby incorporated by reference in their entireties.

After forming the body of the devices described herein, the first chamber can be filled with a viscous solution of the first matrix molecules. The first matrix molecule solution can have a viscosity that is high enough to form a defined structure but also allow a fluid of a lower viscosity to disperse through the viscous solution, e.g., via surface tension-based passive pumping and/or pressure-driven flow, such that a portion of the viscous solution can be removed, thus creating one or more lumens within the viscous solution. In some embodiments, the solution of the first matrix molecules can have a viscosity of about 2 cP to about 40 cP.

In some embodiments, the solution of the first matrix molecules can further comprise tissue-specific and/or blood vessel-associated cells. In some embodiments, tissue-specific and/or blood vessel-associated can be distributed in the first permeable matrix and interact with cells lining the lumen(s). In some embodiments, the lumen (s) can comprise an endothelium on its luminal surface. In some embodiments, the lumen(s) can comprise pericytes covered by an endothelium on its luminal surface. In some embodiments, the lumen(s) can comprise epithelial cells on its luminal surface mimicking a duct or a sinus of a tissue or an organ.

In some embodiments, the method can further comprise forming at least one layer of cells comprising tissue-specific cells and/or blood vessel-associated cells (e.g., fibroblasts, smooth muscle cells, and/or endothelial cells) on the inner surface of the lumen(s). For example, a fluid comprising appropriate cells can be introduced into the lumen(s) such that the cells can adhere on the inner surface of the lumen(s). In some embodiments, the inner surface of the lumen(s) can comprise an endothelial cell monolayer.

In some embodiments, tissue specific cells and/or blood vessel-associated cells can be populated on the second side of the membrane. In these embodiments, the method can further comprise flowing a fluid comprising the tissue-specific cells and/or blood vessel-associated cells through the second chamber such that the cells can adhere on the membrane. In some embodiments, the tissue specific of a second type can be populated in a second permeable matrix disposed in the second chamber. In these embodiments, the method can further comprise forming a second permeable matrix in the second chamber, wherein the second permeable matrix comprises the tissue specific cells of a second type.

In some embodiments, tissue specific cells can be populated on the first side of the membrane. In these embodiments, a fluid comprising the tissue specific cells can be flown through the first chamber, prior to introducing a viscous solution of the first matrix molecules into the first chamber, to allow the cells adhered on the membrane.

Devices for simulating a function of a tissue produced by the methods of making the same are also provided herein.

### Exemplary Methods of Using the Devices and Systems Described Herein

In some embodiments, the device provided in the method can be adapted to any embodiment of the devices described herein.

In some embodiments, the devices described herein can be used to determine an effect of a test agent on the cells on one or both surfaces of the membrane and/or in the first and/or second permeable matrices. Accordingly, in some embodiments, the method can further comprise contacting the tissue-specific cells and/or blood vessel-associated cell layer (e.g., endothelial cell layer) with a test agent.

In some embodiments, the exclusion of fluorescently labeled large molecules (e.g., dextrans of different weight or FITCs) can be quantitated to determine the permeability of the endothelium-lined or pericyte/endothelium-lined lumen(s) and thus assess the barrier function of the epithelium, e.g., in a tissue-specific condition. For example, flowing a fluid containing fluorescently labeled large molecules (e.g., but not limited to, inulin-FITC) into a first chamber cultured with differentiated epithelium can provide a non-invasive barrier measurement. As a functional tight junction barrier will generally prevent large molecules from passing through the epithelium from the first chamber to the second chamber, the absence of the detection of the fluorescently labeled large molecules in the first permeable matrix and in second chamber is generally indicative of a functional barrier function of the epithelium.

The advantages of the devices and systems described herein, as opposed to conventional cell cultures or tissue cultures are numerous. For instance, in contrast to the existing culture models which only allow for culture or co-culture of flat monolayers, the devices described herein allow for more realistic co-culture of at least one or a plurality of (e.g., at least two or more) three-dimensional, endothelium-lined or pericyte/endothelium-lined lumens interacting with tissue specific cells in a more defined three-dimensional architectural tissue-tissue relationships that are closer to the in vivo situation. Thus, cell functions and responses to pharmacological agents or active substances or products can be investigated at the tissue and organ levels.

FIG. 1 illustrates a block diagram of the overall system employing the device in accordance with an embodiment. As shown in FIG. 1, the system 100 includes at least one device described herein for simulating a function of a tissue 102, one or more fluid sources 104, 104n coupled to the device 102, one or more optional pumps 106 coupled to the fluid source 104 and device 102. One or more central processing units (CPUs) 110 can be coupled to the pump 106 and can control the flow of fluid in and out of the device 102. The CPU 110 can include one or processors 112 and one or more local/remote storage memories 114 (including, e.g., a “cloud” system). A display 116 can be optionally coupled to the CPU 110, and one or more external force sources 118 can be optionally coupled to the CPU 110 and the device 102. In some embodiments, the CPU 110 can control the flow direction and/or rate of fluid to the device. It should be noted that although one device 102 is shown and described herein, a plurality of the devices 102 can be tested and analyzed within the system 100 as described herein.

In some embodiments, the devices described herein 102 can include two or more ports which place the first chambers and second chambers of the device 102 in communication with the external components of the system, such as the fluid and external force sources. In particular, the device 102 can be coupled to the one or more fluid sources 104n in which the fluid source can contain air, culture medium, blood, water, cells, compounds, particulates, and/or any other media which are to be delivered to the device 102. In one embodiment, the fluid source 104 can provide fluid to one or more first chambers and second chambers of the device 102. In one embodiment, the fluid source 104 can receive the fluid that exits the device 102. In some embodiments, the fluid exiting the device 102 can additionally or alternatively be collected in a fluid collector or reservoir 108 separated from the fluid source 104. Thus, it is possible that separate fluid sources 104, 104n respectively provide fluid to and remove fluid from the device 102.

One or more sensors 120 can be coupled to the device 102 to monitor one or more areas within the device 102, whereby the sensors 120 provide monitoring data to the CPU 110. In some embodiments, one type of sensor 120 can comprise a force sensor which provides data regarding the amount of force, stress, and/or strain applied to a membrane or pressure in one or more operating channels within the device 102. In one embodiment in which pressure is used within the device, pressure data from opposing sides of the channel walls can be used to calculate real-time pressure differential information between the operating and central sub-channels (e.g., first chambers and second chambers). The monitoring data would be used by the CPU 110 to provide information on the device's operational conditions as well as how the cells are behaving within the device 102 in particular environments in real time. The sensor 120 can be an electrode, have infrared, optical (e.g. camera, LED), or magnetic capabilities or utilize any other appropriate type of technology to provide the monitoring data. For instance, the sensor can be one or more microelectrodes which analyze electrical characteristics across the membrane (e.g. potential difference, resistance, and short circuit current) to confirm the formation of an organized barrier, as well as its fluid/ion transport function across the membrane. It should be noted that the sensor 120 can be external to the device 102 or be integrated within the device 102. In some embodiments, the CPU 110 controls operation of the sensor 120, although it is not necessary. The data can be shown on the display 116.

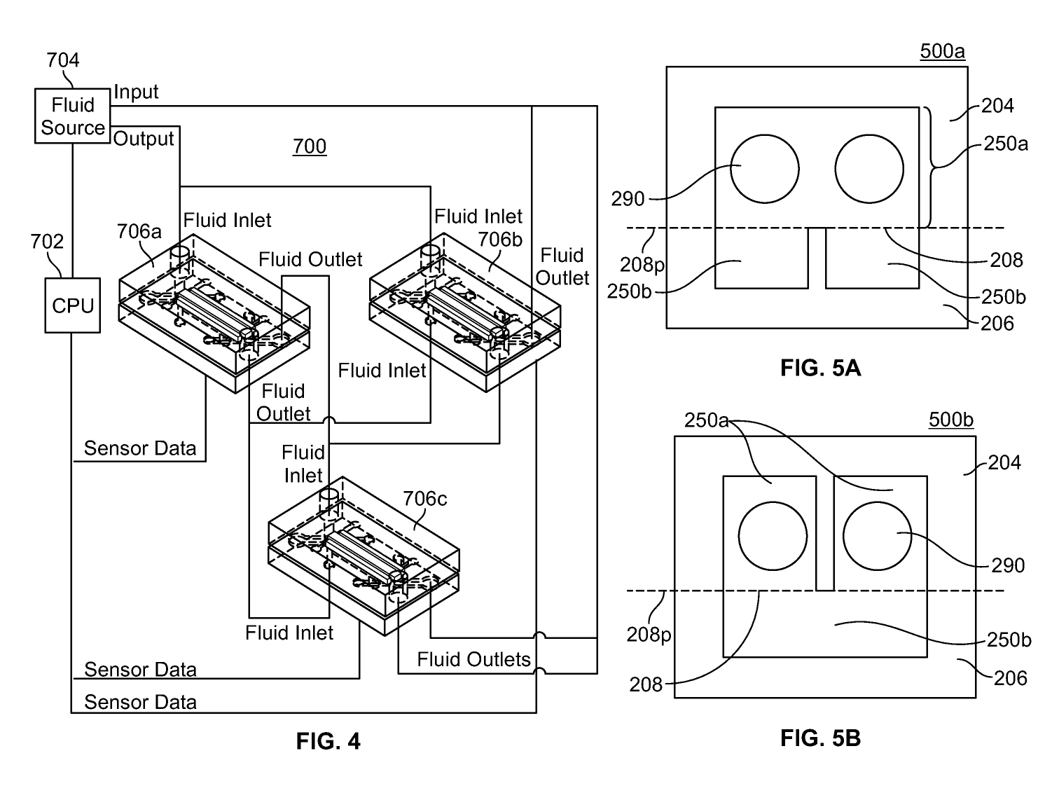

FIG. 4 illustrates a schematic of a system having at least one device 706A in accordance with an embodiment described hereinfluidically connected to another device 706B described herein and/or any cell culture device known in the art, e.g., an art-recognized organ-on-a-chip 706C. As shown in FIG. 4, the system 700 includes one or more CPUs 702 coupled to one or more fluid sources 704 and external force sources (e.g., pressure sources) (not shown), whereby the preceding are coupled to the three devices 706A, 706B, and 706C. It should be noted that although three devices 706 are shown in this embodiment, fewer or greater than three devices 706 can be used. In the system 700, two of the three devices (i.e., 706A and 706B) are connected in parallel with respect to the fluid source 704, and two of the three devices (i.e., 706A and 706C) are connected in serial fashion with respect to the fluid source 704. It should be noted that the shown configuration is only one example and any other types of connection patterns can be utilized depending on the application. In some embodiments, a system can be the one described in the International Patent Application No. PCT/US12/68725, titled “Integrated Human Organ-on-Chip Microphysiological Systems,” where one or more devices described herein can be fluidically connected to form the system.

It should be understood that this invention is not limited to the particular methodology, protocols, and reagents, etc., described herein and, as such, can vary. The terminology used herein is for the purpose of describing particular embodiments only, and is not intended to limit the scope of the present invention, which is defined by the claims.

Other than in the operating examples, or where otherwise indicated, all numbers expressing quantities of ingredients or reaction conditions used herein should be understood as modified in all instances by the term “about.” The term “about” when used to described the present invention in connection with percentages means ±5%.

In one aspect, the present invention relates to the herein described compositions, methods, and respective component(s) thereof, as essential to the invention, yet is open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of'). This applies equally to steps within a described method as well as compositions and components therein. In other embodiments, the inventions, compositions, methods, and respective components thereof, described herein are intended to be exclusive of any element not deemed an essential element to the component, composition or method (”consisting of').

All patents, patent applications, and publications identified are expressly incorporated herein by reference for the purpose of describing and disclosing, for example, the methodologies described in such publications that might be used in connection with the embodiments and methods described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

The following examples illustrate some embodiments and aspects described herein. It will be apparent to those skilled in the relevant art that various modifications, additions, substitutions, and the like can be performed without altering the spirit or scope of the invention, and such modifications and variations are encompassed within the scope of the invention as defined in the claims that follow. The following examples do not in any way limit the invention.

### Example 1

### Simulation of a Blood-Brain-Barrier Using One Embodiment of the Devices Described Herein

This Example illustrates an in vitro model of a blood-brain barrier using one embodiment of the devices described herein, e.g., as shown in FIG. 2A, cultured with cells from a neurovascular and a micropatterned extracellular matrix. As used herein, the term “micropatterned” refers to a permeable matrix or scaffold material comprising at least one or more (including, e.g., at least two, at least three, at least four, at least five, at least six or more) lumens. In some embodiments, the matrix or scaffold material can comprise a gel or hydrogel. In some embodiments, the device comprises (i) a first structure defining a first channel, the first channel comprising a first permeable matrix disposed therein, wherein the first permeable matrix comprises at least one or a plurality of (e.g., at least two or more) lumens each extending therethrough; (ii) a second structure defining a second channel; (iii) a membrane located at an interface region between the first structure and the second structure to separate the first channel from the second channel, the membrane including a first side facing toward the first channel and a second side facing toward the second channel.

In some embodiments, the first channel can have a width and/or height of about 1 mm and a length of about 2 cm, and the second channel can have a width of about 1 mm, a height of about 200 μm, and a length of about 2 cm.

In some embodiments, the two channels are separated by a porous membrane (e.g., a porous PDMS membrane) with a thickness of about 50 μm and pores of about 7 microns in diameter.

To create the blood-brain-barrier device, at least one or more endothelial cell-lined or pericyte/endothelial cell-lined lumens can be formed in the first permeable matrix disposed in the first channel.

In some embodiments, the first channel can be filled with a pericyte-containing viscous solution of collagen I (e.g., at a concentration of about 5 mg/ml). It is contemplated that other gels of proteins and synthetic material may also be used including, but not limited to, MATRIGEL®, high concentration laminin, fibrin gels, pluronic gel, porous plastic materials, polymeric matrices, or any combination thereof. One or more circular lumens can be created in the collagen I viscous solution. In some embodiments where a high protein concentration can be limiting or interfering with cellular processes (e.g., migration, growth, and/or extension of processes) of cells embedded therein, a protein molecule such as an extracellular matrix molecule (e.g., collagen and/or laminin) at a lower concentration may be mixed with a viscosity modifier (e.g., PEG) to achieve a high viscosity. At least one pressure-driven flow of a fluid with a lower viscosity can then be generated in the viscous solution to pattern one or more generally circular lumens in the highly viscous solution. After gelation of the viscous matrix solution at about 37 degrees, the patterned lumen(s) can be populated with endothelial cells or sequentially with pericytes and endothelial cells, to generate endothelialized tube(s) with an open lumen. Thus, in some embodiments, the lumen(s) can be lined with an endothelium. In some embodiments, the lumen(s) can be lined with pericytes covered by an endothelium.

In some embodiments, the second channel can be populated with astrocytes and neurons. In some embodiments, astrocytes can be cultured on the side of the membrane facing the second channel. The second channel can then be infused with a neuronal cell suspension, e.g., in MATRIGEL®, and the cell-containing gel suspension is allowed to gel. In some embodiments, the concentration of the MATRIGEL® can range from about 5 mg/mL to about 11 mg/mL.

Thus, by controlled patterning of cell types and matrices in the channels separated by a membrane, a blood-brain barrier-on-a-chip, which is a neurovascular co-culture with an organization that is highly reminiscent of the organization of the neurovascular unit in vivo, can be generated. Endothelial cells face an open lumen and interact with a matrix containing pericytes on their basal side, while a layer of astrocytes separates the perivascular gel from a neuronal compartment in which neurons grow and interact to form a neuronal network. As such, the blood-brain barrier-on-a-chip as described herein can provide a generally versatile and realistic setting to perform predictive studies of blood-brain barrier function and transport.

In some aspects, the devices described herein combine creation of a three-dimensional hollow structure in an extracellular matrix protein gel by viscous fingering with compartmentalization of different cell types by one or multiple synthetic membranes. Such design can allow for a controlled and physiologically realistic co-culture of endothelialized lumen(s) with monolayers and/or three-dimensional cultures. For example, in some embodiments, the design can allow for realistic co-culture of endothelium, pericytes, astrocytes and neurons in a configuration and in a matrix that is more realistic than what can be achieved with existing Transwell or microfluidic blood-brain barrier models, which only allow for co-culture of flat monolayers. In addition, the devices described herein can permit innervation of neurites from one chamber to another chamber.

In some embodiments, the cells in the devices described herein can be exposed to one or more exogenous stimuli, e.g., pro-inflammatory agents. As used herein, the term “pro-inflammatory agent” refers to an agent that can directly or indirectly induce or mediate an inflammatory response in cells, or is directly or indirectly involved in production of a mediator of inflammation. A variety of proinflammatory agents are known to those skilled in the art. Illustratively, pro-inflammatory agents include, without limitation, eicosanoids such as, for example, prostaglandins (e.g., PGE2) and leukotrienes (e.g., LTB4); gases (e.g., nitric oxide (NO)); enzymes (e.g., phospholipases, inducible nitric oxide synthase (iNOS), COX-1 and COX-2); and cytokines such as, for example, interleukins (e.g., IL-1α, IL-1β, IL-2, IL-3, IL-4, IL-5, IL-6, IL-8, IL-10, IL-12 and IL-18), members of the tumor necrosis factor family (e.g., TNF-α, TNF-β and lymphotoxin β, interferons (e.g., IFN-β and IFN-γ), granulocyte/macrophage colony-stimulating factor (GM-CSF), transforming growth factors (e.g., TGF-β1, TGF-β2 and TGF-β3, leukemia inhibitory factor (LTF), ciliary neurotrophic factor (CNTF), migration inhibitory factor (MTF), monocyte chemoattractant protein (MCP-I), macrophage inflammatory proteins (e.g., MIP-1α, MIP-1β and MIP-2), and RANTES, as well as environmental or physical agents such as silica micro- and nano-particles and pathogens. In some embodiments, at least one or more of these pro-inflammatory agents can be added to a cell culture medium, e.g., to stimulate or challenge the cells within the device to simulate an inflammatory response or an inflammation-associated disease, disorder, or injury in vivo.

### Example 2

### Simulation of a Blood-Brain-Barrier using One Embodiment of the Devices Described Herein.

As discussed above, neurovascular dysfunction is of major importance in the pathophysiology of neurological disorders, but modeling these processes in vitro has proven to be difficult due to the complex multicellular, three-dimensional organization of blood vessels in the brain. This Example illustrates three-dimensional microcultures of human neurovascular cell types that closely resemble the organization of the blood vessels of the brain in vivo. The model can be established by seeding cells in and around a circular lumen that is patterned or created inside a collagen gel. In some embodiments, human astrocytes can be embedded in the collagen gel prior to the three-dimensional patterning. In some embodiments, human brain pericytes can be seeded inside a patterned lumen, and human cerebral cortex microvascular endothelial cells can then be used to cover the entire lumen with a monolayer. Thus, three-dimensional co-cultures between relevant neurovascular cell types inside may be established in a microfluidic device.

To create such three-dimensional microdevices mimicking the blood-brain-barrier, in some embodiments, a device comprising a channel may be filled with viscous solution of collagen I (e.g., at a concentration of about 5 mg/ml). A circular lumen may be created in the collagen I. Methods to create lumens in permeable matrices or scaffolds are generally known in the art. For example, a pressure-driven flow of a fluid with a viscosity lower than that of the viscous solution of collagen I may be used to pattern a generally circular lumen in the viscous solution. In some embodiments, human primary astrocytes may be dispersed in the collagen I solution. After gelation of the collagen I solution at about 37 degrees, the patterned lumen may be populated by endothelial cells to generate an endothelialized tube with an open lumen. Alternatively, the lumen may be sequentially populated with pericytes and endothelial cells to generate a pericyte/endothelium-lined tube with an open lumen, where the endothelium covers the pericytes. The devices were kept in culture to allow the endothelial cells to form a monolayer with tight junctions.

In some embodiments, the devices described herein can be used to study cytokine release. For example, cytokine release in the microdevice was compared to Transwell systems. Transwell inserts were populated with pericytes or astrocytes on the basal side of the permeable membrane and endothelial cells on the apical side of the membrane. The Transwells were then kept in culture to allow the endothelial cells to form a monolayer with tight junctions.

Following overnight starvation in low serum cell culture medium, the microdevices or Transwells were exposed to an inflammatory stimuli (e.g., TNF-alpha at a concentration of about 50 ng/mL) or control conditions for about 6 hours. Cytokine secretion was thereafter collected for about 1 hour under flow in microdevices (e.g., at a flow rate of about 0.1 mL/hr) and under static conditions in Transwells. Cytokine release was quantified by a BIO-PLEX® Pro Cytokine kit from Bio-Rad Laboratories (Hercules, Calif., USA). Experiments were performed as 3-5 replicates for each condition and normalized to cytokine release from endothelial monoculture device or Transwell.

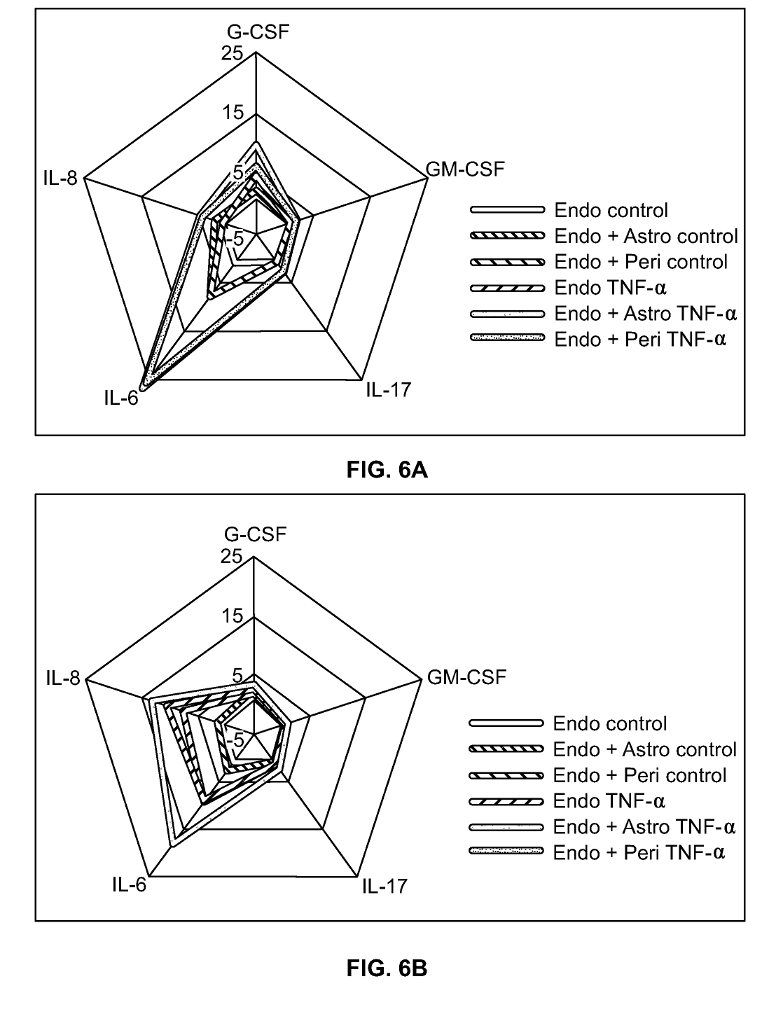

The cytokine release profile (comprising, e.g., G-CSF, GM-CSF, IL-17, IL-6, and IL-8) in these three-dimensional microcultures was compared with conventional Transwell cultures after inflammatory stimuli. FIGS. 6A-6B shows data graphs showing cytokine release profiles in various systems normalized to unstimulated devices with an endothelial culture. As shown in FIGS. 6A-6B, there were significant differences in the cytokine release profile between these two in vitro models of the neurovascular unit, showing that the three-dimensional microcultures provide different cellular interaction dynamics from the conventional Transwell cultures.

### Other Examples

According to embodiments described herein, a three-dimensional (3D) model of the human blood-brain barrier (BBB) was microengineered within a microfluidic chip by creating a generally cylindrical collagen gel containing a generally central hollow lumen inside a microchannel, culturing primary human brain microvascular endothelial cells on the gel's inner surface, and flowing medium through the lumen. Studies were carried out with the engineered microvessel containing endothelium in the presence or absence of either primary human brain pericytes beneath the endothelium or primary human brain astrocytes within the surrounding collagen gel to explore the ability of this simplified model to identify distinct contributions of these supporting cells to the neuroinflammatory response. This human 3D blood-brain-barrier-on-a-chip exhibited barrier permeability similar to that observed in other in vitro blood-brain barrier (BBB) models created with non-human cells, and when stimulated with the inflammatory trigger, tumor necrosis factor-alpha (TNF-α), different secretion profiles for granulocyte colony-stimulating factor (G-CSF) and interleukin-6 (IL-6) were observed, depending on the presence of astrocytes or pericytes. Importantly, the levels of these responses detected in the 3D BBB chip were significantly greater than when the same cells were co-cultured in static Transwell plates. Thus, as G-CSF and IL-6 have been reported to play important roles in neuroprotection and neuroactivation in vivo, the 3D BBB chip described herein offers a new method to study human neurovascular function and inflammation in vitro and to identify physiological contributions of individual cell types.

In the following examples, an in vitro model of the human BBB was developed that would permit analysis of the independent contributions of human brain microvascular endothelium, pericytes, and astrocytes to the response of the BBB to inflammation stimuli. The inflammatory effects of various stimuli, including TNF-α, lipopolysaccharide (LPS) endotoxin, nanoparticles, and HIV-virions have been studied previously using static BBB models with non-human and human cells cultured in Transwell plates. Studies with these models have also demonstrated that both astrocytes and pericytes can influence the barrier function of the BBB under static conditions. But given inevitable species differences between humans and animal models in terms of species-specific efflux transporter activity, tight junction functionality and cell-cell signaling, it is important to carry out studies using normal human brain microvascular cells to recapitulate human brain microvascular physiology. In fact, interactions between human primary astrocyte and human brain microvascular cells have been analyzed in static Transwell cultures, and the results of these studies have shown correlations with in vivo studies for radiotracer permeability profiles and barrier function. However, hemodynamic forces and the physical tissue microenvironment are also known to contribute significantly to microvascular function. Thus, to best model the BBB in vitro, it is important to mimic these key physical features of the brain capillary microenvironment, including fluid flow, extracellular matrix (ECM) mechanics and the cylindrical geometry of normal brain microvessels. BBB cell culture models based on semi-permeable, synthetic hollow-fibers with a blood vessel-like geometry and fluid flow have been developed, and more recently, microfluidic models of the BBB have been reported that enable co-culture of endothelium with pericytes, astrocytes, or neurons while being exposed to fluid flow and low levels shear stress. However, all of these in vitro BBB models utilized rigid ECM substrates that have stiffness values orders of magnitude higher than those observed in living brain microvessles (i.e., about 1 GPa for ECM-coated cell culture plastic versus about 1 kPa in vivo) and none cultured neurovascular cells in a normal cylindrical vascular conformation. Microfluidic models have been developed that contain more flexible ECM gels and reconstitute 3D hollow vessel-like structures, but the only reported studies that use such techniques to model the BBB used non-human endothelium. Human brain endothelial cells, pericytes, and astrocytes also have been maintained in close juxtaposition in spheroid cultures, but vessels do not form in these structures, and instead, they resemble endothelium-lined spheres. In the following examples, a 3D microfluidic model of a hollow human brain microvessel was developed that contains closely apposed primary microvascular endothelial cells, pericytes, and astrocytes isolated from human brain, specifically to analyze the contribution of the individual cell types to neurovascular responses to inflammatory stimuli. The utility of this new organ-on-a-chip model for studying neurovascular inflammation was demonstrated by measuring cytokine release induced by adding tumor necrosis factor-alpha (TNF-α) as an inflammatory stimulus, and analyzing how the presence of astrocytes and pericytes independently contribute to this response. As this 3D BBB-on-a-chip permits analysis of the contributions of individual cell types to neuropathophysiology, it may be useful for studies focused on the mechanisms that underlie inflammation in the human brain as well as related screening of neuroactive therapeutics.

Materials and Methods

Cell Culture

Human brain microvascular endothelial cells (hBMVECs) and human brain pericytes, both derived from cortex, were obtained from Cell Systems (Kirkland, Wash., USA) and maintained with CSC complete medium (Cell Systems) on regular tissue culture flasks coated with an attachment factor (Cell Systems). Human astrocytes of cortical origin were obtained from ScienCell (San Diego, Calif., USA) and maintained in Astrocyte medium (ScienCell). All cells were used at passage 3 to 8.

Microfluidic Chips, Fabrication and Pre-Treatment

Molds for microfluidic channels with a width, height, and length of about 1 mm, about 1 mm, and about 20 mm, respectively, were designed with SOLIDWORKS® software (Dassault Systèmes SolidWorks Corp. (Concord, Mass., USA)) and produced by FINELINE® stereolithography (Proto Labs, Inc. (Maple Plain, Minn., USA)). Microfluidic devices were subsequently produced by soft lithography. Briefly, a degassed 10:1 base:crosslinking mix of Sylgard 184 polydimethylsiloxane (PDMS, Dow Corning, Inc. (Midland, Mich., USA)) was poured onto the mold and allowed to crosslink at about 80° C. for about 18 hours. Inlets and outlets of about 1.5 mm diameter were punched in the molded PDMS and the device was bonded to an about 100 μm layer of spincoated PDMS by pre-treating with oxygen plasma at about 50 W for about 20 seconds in a PFE-100 (Plasma Etch, Inc. (Carson City, Nev., USA)) and then pressing the surfaces together. After baking at about 80° C. for about 18 hours, devices were again treated with oxygen plasma (about 30 seconds, about 50 W) and silanized by immediately filling them with about 10% (v/v) of (3-aminopropyl)-trimethoxysilane (Sigma-Aldrich (St. Louis, Mo., USA)) in about 100% ethanol and incubating at room temperature for about 15 minutes. Devices were then flushed with about 100% ethanol, followed by water and ethanol and subsequently dried at about 80° C. for about 2 hours. Subsequently, the surfaces were further functionalized by filling the devices with about 2.5% glutaraldehyde (Electron Microscopy Services, Inc.). After incubating for about 15 minutes, the devices were rinsed extensively with deionized water and ethanol and were baked for about 2 hours at about 80° C. The Schiff bases formed on proteins after glutaraldehyde immobilization were stable without further reduction, as has been demonstrated in surface-protein conjugation.

Viscous Fingering to Generate Lumens in Collagen Gels

The viscous fingering procedure was performed as previously reported, with slight modifications. To minimize delamination of the collagen gel tended to delaminate from the PDMS microchannel surface, the PDMS surface was functionalized in a three-step process involving oxygen plasma treatment, amino-silane conjugation, and glutaraldehyde derivatization. This treatment improved the stability of the PDMS-collagen interaction such that generally no delamination was observed, and this protocol allowed the chips to remain stable for more than 7 days with no apparent degradation.

All devices pre-treated in this manner were kept on ice and filled with about 5 mg/ml of ice cold rat tail collagen I (Corning), mixed and neutralized as per the manufacturer's instructions. After filling the device with the collagen solution, a 200 μl pipette tip with about 100 μl of ice-cold culture medium was inserted in the inlet. The medium was allowed to flow through the viscous collagen solution by hydrostatically driven flow and the devices were subsequently incubated at about 37° C. to allow the formation of collagen gels. Alternatively, to correlate hydrostatic pressure with lumen diameter, the devices were connected to a liquid reservoir that could be placed at different heights. The pressure values presented were calculated as the difference in height between the meniscus of the liquid in the reservoir and the inlet of the chip. After collagen gelation by incubating for about 30 minutes at about 37° C., the devices were rinsed extensively with pre-warmed culture medium and stored in a cell culture incubator for about 18 hours. An input pressure of about 2.6 cm H₂O (about 0.26 kPa) was used to form the lumen, and a minimal pressure of about 1.5 cm H₂O (about 0.15 kPa) was needed to initiate formation of the finger in a collagen gel in the about 1×1 mm channel. Microchannels with smaller dimensions, down to about 300×300 μm were evaluated, but these yielded significantly lower success rates due to increased clogging of lumens with collagen or complete removal of the gels due to the need to apply increased pressures.

Cell Culture in Three-Dimensional Gels

Human astrocytes were incorporated in the bulk of the collagen by mixing in a final concentration of about 3×10⁶ cells/ml in the gel. Following about 18 hours of incubation of devices in a cell culture incubator, sequential seeding of pericytes and hBMVECs was carried out to line the cylindrical lumen with these two cells types. Pericytes were seeded into the devices at about 0.8×10⁶ cells/ml in two rounds, where the devices were put upside down in the first seeding round. An incubation period of about 30 min was allowed between the seeding steps. About 30 minutes after pericyte seeding hBMVECs were seeded at about 2.4×10⁶ cells/ml under flow for about 20 seconds (about 120 μl/min; about 1 dyne/cm² shear stress) using the described two-step seeding method to obtain a lumen lined with an endothelial monolayer. About one hour after final cell seeding, medium was exchanged by hydrostatically driven flow. The chips were maintained under static conditions in a cell culture incubator with the cell culture medium being exchanged over a period of about 5 minutes every about 24 hours using hydrostatically-driven flow at about 120 μl/min (about 1 dyne/cm² shear stress). Once a confluent monolayer formed, which was typically after about 72 hours, about 250 μM of a cell-permeable cyclic adenosine monophosphate, 8-CPT-cAMP (Abcam (Cambridge, Mass., USA)) and about 17.4 μM of the phosphodiesterase inhibitor Ro 20-1724 (Santa Cruz Biotech (Dallas, Tex., USA)) was added to the medium, which was exchanged periodically as described above. The cells were not cultured under continuous flow for the about 5 days of culture because, to get a realistic shear stress in the range of about 1-10 dyne/cm², flow rates in the range of 600 ml/hour would be needed, which would be cost-prohibitive.

Permeability Assay

TEER could not be measured to evaluate the barrier function of the 3D BBB chip due to the difficulty of placing electrodes on opposite sides of the endothelium with a surrounding solid ECM gel and ensuring an even electrical field given the device geometry. Instead, the permeability coefficient for small molecular (3 kDa) fluorescent dextran was evaluated. Devices were cultured for about 120 hours before they were mounted on a Zeiss AXIO® Observer microscope (Carl Zeiss AG Corp., Oberkochen, Germany), with a 5× air objective, numerical aperture 0.14 with an EVOLVE™ EMCCD camera (Photometrics (Tuscon, Ariz., USA)). Culture medium with about 5 μg/ml dextran 3 kDa-Alexa488 (Life Technologies (Beverly, Mass., USA)) was continuously infused in the microfluidic chips at about 5 ml/hour with a syringe pump (about 0.7 dyne/cm² shear stress) and fluorescent images were recorded about every 3 seconds over 2 hours. Apparent permeability (Pₐₚₚ) was calculated by analyzing total fluorescence intensity in an area of about 1 mm by about 1 mm and then applying Pₐₚₚ=(1/ΔI) (dI/dt)₀ (r/2), where ΔI is the increase in total fluorescence intensity upon adding labeled dextran, (dI/dt)₀ is the initial rate of increase in intensity as dextran diffuses out of the tube into the surrounding gel, and r is the radius of the tube. (dI/dt)₀ was determined by analyzing the linear increase in fluorescence signal during about 5 minutes. Control measurements for the recorded intensity demonstrated a linear response of the detector in the range of about 5 μg/ml dextran 3 kDa-Alexa488. The wide depth of field of the objective allowed for collection of all fluorescent signal from the about 1 mm high channel. Control measurements confirmed that the fluorescence signal from microchannels of heights of about 200 μm-1000 μm filled with about 5 μg/ml dextran 3 kDa-Alexa488 increased linearly with channel height. The permeability measurement method cannot be applied to the bare collagen lumens or to cultures of astrocytes or pericytes alone because the diffusion of the 3 kDa dextran is too fast to reliably establish the intensity step ΔI.

Transwell Cell Culture

24-well Transwell inserts (Corning), about 0.4 μm, polyethylene terephthalate membranes, were coated with rat-tail collagen I (Corning) at about 100 μg/ml in phosphate-buffered saline for about 2 hours. The inserts were inverted and pericytes or astrocytes were seeded at about 6.25×10³ cells per insert. After about 2 hours of incubation, the inserts were placed in 24-well plates and seeded with hBMVEC at about 2.5×10⁴ cells per insert. Transendothelial electrical resistance (TEER) values were measured after about 120 hrs of culture using an EndOhm (WPI) and chopstick electrodes. Paracellular diffusion was assayed about 5 minutes after adding dextran 3 kDa-Alexa488 (about 100 μg/ml) to the apical chamber and using a Synergy Neo platereader (BioTek (Winooski, Vt., USA)).

Inflammatory Stimulation and Analysis of Cytokine Release

Microfluidic chips and Transwell inserts were cultured for about 72 hours, followed by incubation in CSC complete medium with fetal bovine serum reduced from about 10% to about 2% for about 18 hours. Microfluidics chips were stimulated with TNF-α (Sigma-Aldrich) at about 50 ng/ml in CSC complete medium with about 2% serum for about 6 hrs (about 5 min flow at about 120 μl/min corresponding to about 1 dyne/cm², followed by static conditions). Transwells were stimulated on the apical and the basal side. Following thorough rinsing of microfluidic chips under continuous flow (about 120 μl/min, about 1 dyne/cm²) and batch washes of Transwell plates with CSC complete medium with about 2% serum, conditioned medium from the chips was collected continuously for about 1 hour at about 100 μl/hr (about 0.01 dyne/cm²) using syringe-driven flow; medium from the apical compartment was collected from Transwells after about 1 hour. The cytokine release profile was assayed with the Bio-Plex Pro Human Cytokine 17-plex Assay (Bio-Rad) in a Bioplex 3D system (Bio-Rad), and the resulting cytokine release profiles were normalized to cell culture area in 3D BBB chips versus Transwells.

Fixation, Staining and Imaging

Microfluidic chips were cultured for about 96 hours followed by rinsing in phosphate-buffered saline and fixation in about 4% paraformaldehyde (Sigma) for about 20 minutes at room temperature. Cell-free devices were fixed about 30 minutes after collagen gelation. Immunocytochemistry was carried out after permeabilization in phosphate-buffered saline with about 0.1% Triton X-100 (Sigma) and blocking for about 30 minutes in about 10% goat serum in phosphate-buffered saline with about 0.1% Triton-X 100. The following primary antibodies were used for immunocytochemistry experiments: rabbit anti-glial fibrillary acidic protein (GFAP) (EMD Millipore HQ (Billerica, Mass., USA), 1:100), mouse anti-vascular endothelial (VE)-cadherin (Abcam (Cambridge, Mass., USA), 1:100), mouse anti-PECAM (eBiosciences (San Diego, Calif., USA), 1:100), mouse anti-zona occludens-1 (ZO-1) (Invitrogen (Carlsbad, Calif.), 1:100), rabbit anti-alpha-smooth muscle actin (SMA) (Sigma, 1:100) and mouse anti-collagen IV (EMD Millipore). The secondary antibodies were anti-rabbit or anti-mouse IgG conjugated with Alexa Fluor-488, Alexa Fluor-555, or Alexa Fluor-647 (Invitrogen). Hoechst (about 10 mg/ml, Invitrogen) was used at a dilution of about 1:5000 for nuclei staining. For staining of F-actin, Alexa Fluor-488-phalloidin or Alexa Fluor-647-phalloidin (Invitrogen) were used at dilution of about 1:30. Imaging was carried out using a Leica SP5 X MP Inverted Laser Scanning Confocal Microscope with a 25× water immersion objective and a Zeiss Axio Observer microscope. Conventional confocal imaging was carried out with a 405 laser diode, an Argon laser and a tunable white laser. Second harmonic generation was carried out using two-photon excitation at about 810 nm and detecting emitted light through an about 400-410 nm bandpass filter. Image processing was done using Huygens deconvolution and stitching for tiled images (SVI), Imaris (Bitplane) and Image. The low objective flatness gives a Gaussian intensity profile over each recorded image, which becomes apparent in stitched images 2c and 2k.

Statistics

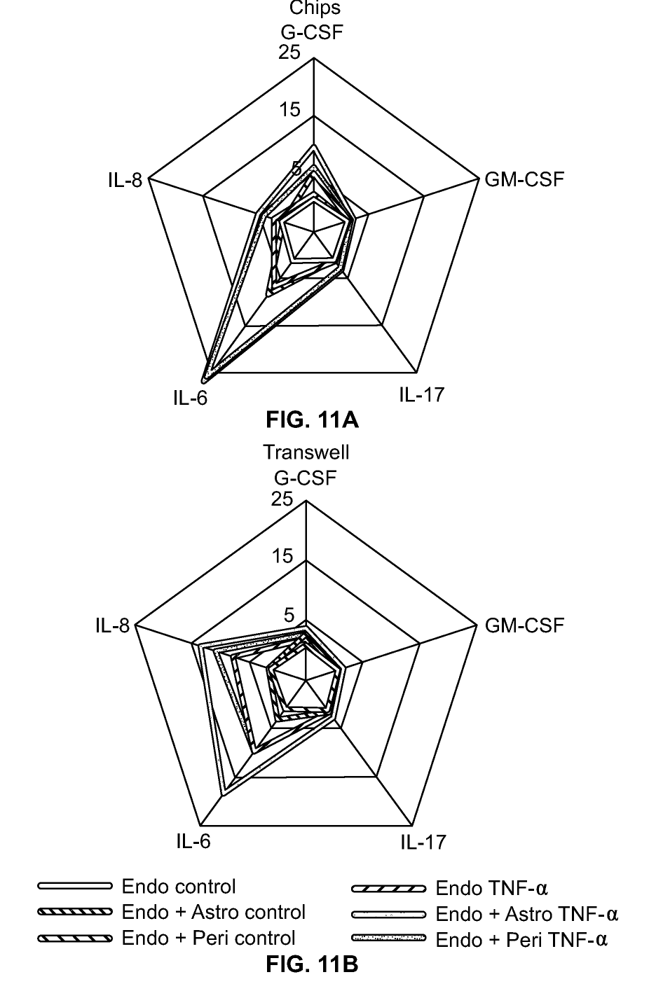

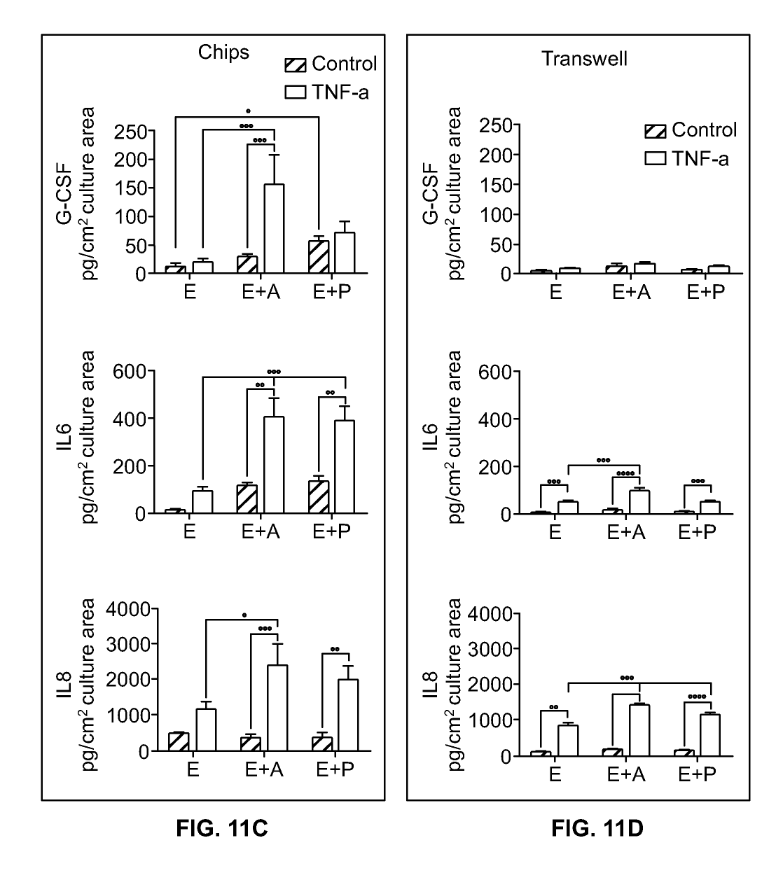

All experiments were carried out at n=3-7. Prism (GraphPad) was used for one-way ANOVA analysis with Bonferroni post-test. **** denotes p<0.0001, *** denotes 0.0001<p<0.001, ** denotes 0.001<p<0.01, * denotes 0.01<p<0.05 (see FIGS. 11C, 11D). For significance testing between two conditions, a non-paired student's t-test was used.

### Results and Discussion

Engineering of the 3D BBB Chip

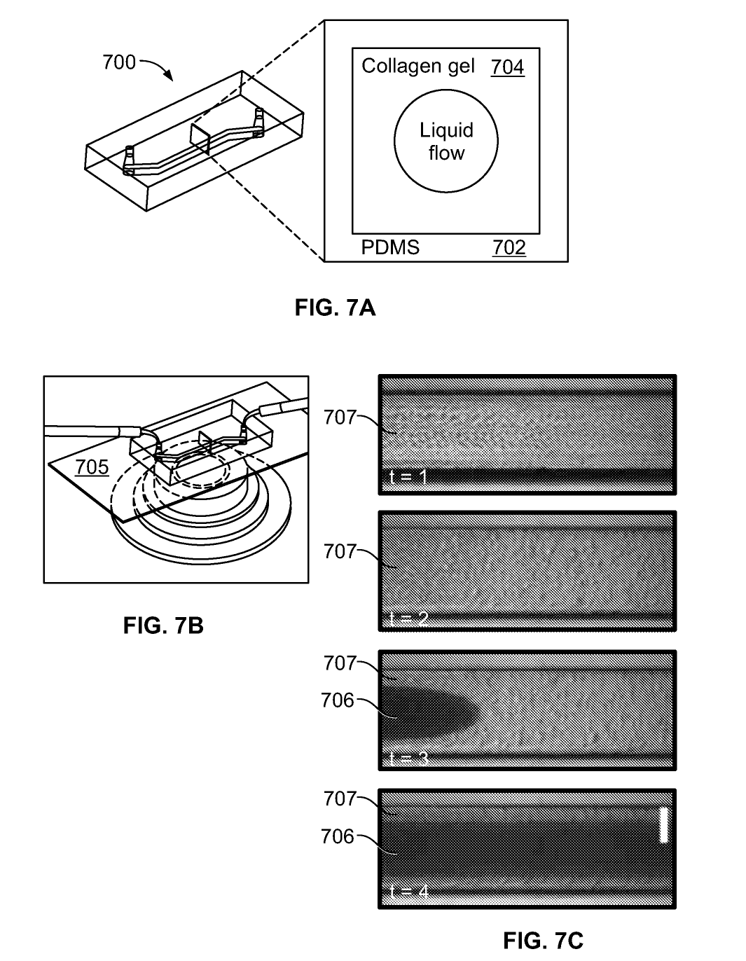

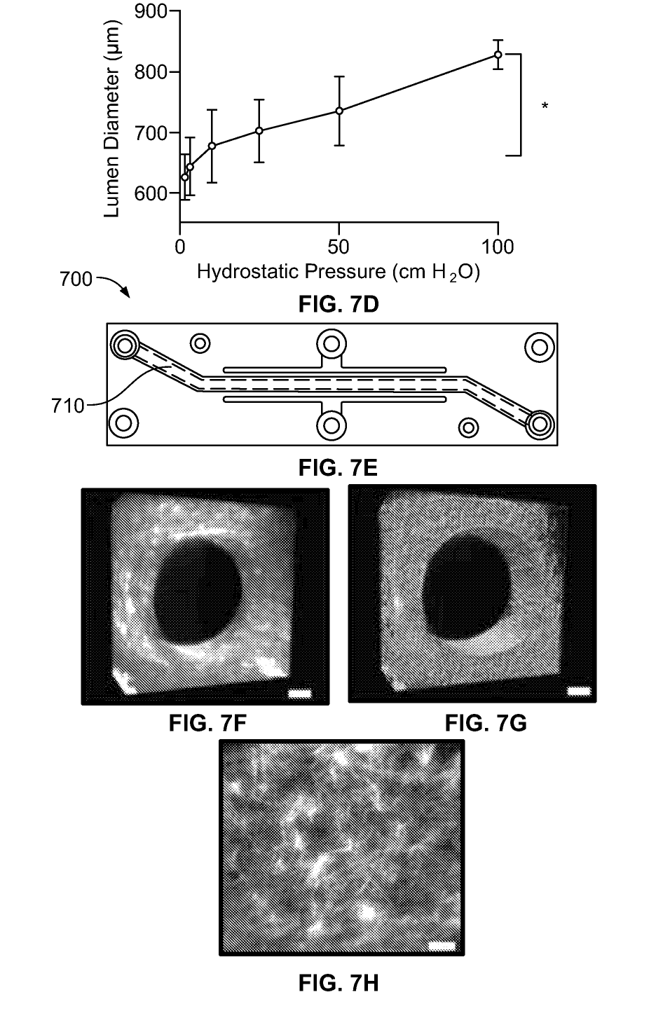

Referring to FIGS. 7A and 7B, to build a 3D BBB chip containing a hollow endothelium-lined microvessel surrounded by a compliant ECM, a cylindrical collagen gel 704 was formed within a single square-shaped microchannel (about 1 mm high×about 1 mm wide×about 2 cm long) (FIG. 7A) in an optically clear polydimethysiloxane (PDMS) chip mounted on a standard glass microscope slide 705 (FIG. 7B) using soft lithography, as previously described. The generally cylindrical collagen gel 704 was formed using a viscous fingering method by first filling the channel with a solution of type I collagen (about 5 mg/ml), applying hydrostatically-controlled medium flow (by varying the height of the fluid reservoir) to finger through the viscous solution, and incubating the chips at about 37° C. to promote gelation (see FIG. 7C). The entire process took about 30 seconds and resulted in the creation of a well-defined lumen with a diameter of about 600 to about 800 μm protruding all the way through the about 2 cm long channel of the microfluidic chip (FIG. 7E). The dimensions of the lumen are controlled by the channel dimensions and by the differences in viscosity and density between the displacing and displaced liquid. Theoretically, an increased pressure will produce a higher tip velocity of the finger, which should lead to a narrower finger (smaller lumen diameter); however, it was empirically found that progressively increasing the hydrostatic pressure of the injected medium resulted in a concomitant increase in lumen diameter, as shown in FIG. 7D. It is possible that the positive correlation between the observed input pressure and lumen diameter might be due to increased shearing of collagen at high flow velocities in the channel directly after the lumen has formed.

Use of second harmonic generation imaging revealed that the cylindrical collagen gel formed in the microchannel with this viscous fingering method contained a generally homogenous, loose, fibrillar collagen matrix with a low number of points of high fibril density located preferentially along the wall of the PDMS channel, as shown in FIGS. 7F-7H. This loose, homogenous ECM network is more similar to that observed in the subendothelial space in the brain than the planar ECM-coated substrates used in past BBB chip models. In addition, when supporting cells, such as human brain astrocytes, are suspended into the collagen solution, they generally evenly distribute throughout the gel as it undergoes viscous fingering and gelation in the microchannel (FIG. 7C). Thus, this cylindrical collagen gel is generally well suited to recapitulate the supporting ECM framework of the BBB on-chip. Moreover, the viscous fingering or other lumen formation methods in hydrogels could be used to further explore the contributions of ECM composition and mechanics in future studies.

Structural Reconstitution of the Human Blood-Brain Barrier

To mimic the human BBB in vitro, primary human brain-derived microvascular endothelial cells were seeded on the inner surface of the cylindrical collagen gel by flowing about 40 μl of a cell suspension through the lumen, stopping flow for about 1 hour to allow them to attach, and then reconstituting medium flow for about 5 min at a shear stress of about 1 dyne/cm² once every day over about 4-5 days of culture.

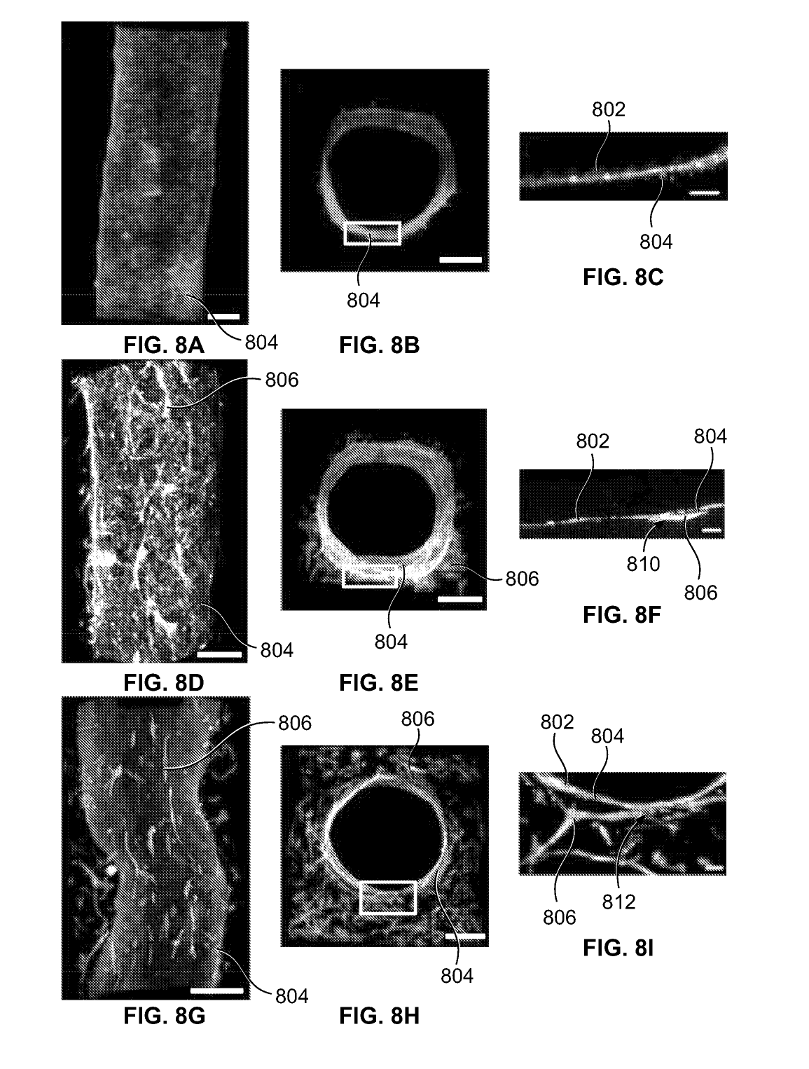

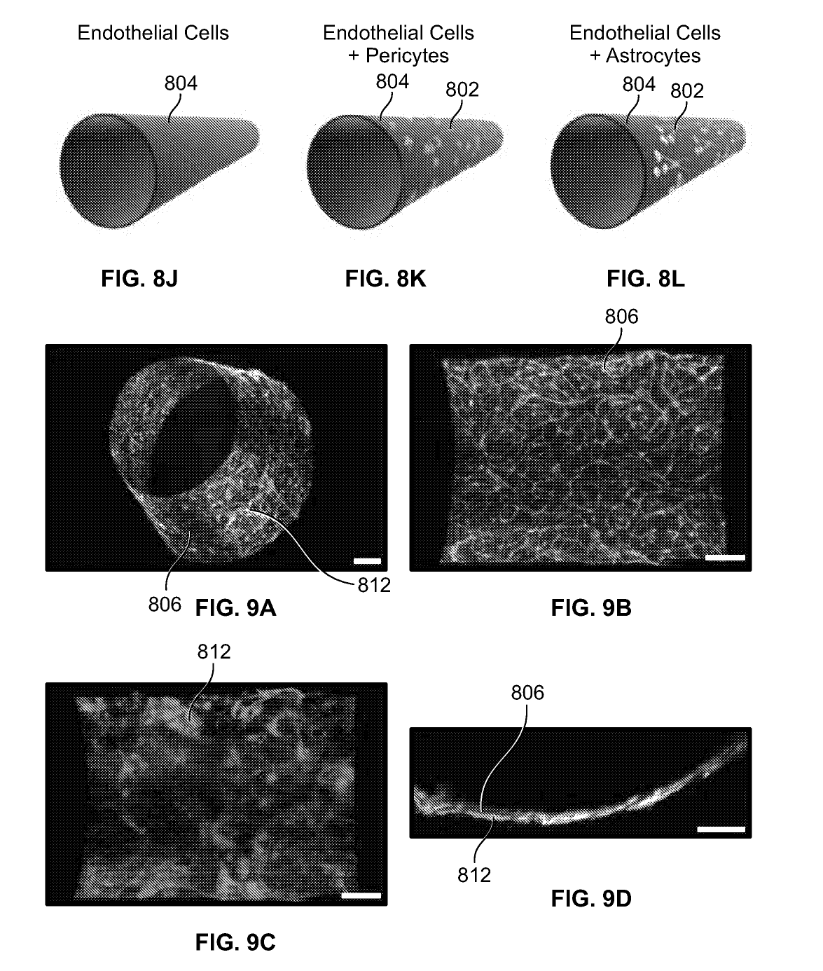

FIGS. 8A-L illustrate co-cultures of human brain microvascular endothelial cells, pericytes, and astrocytes in a 3D BBB chip. Schematic illustrations of the cells populating the 3D vessel structures for three experimental set-ups are shown in FIGS. 8J-8L, and fluorescence confocal micrographs of the engineered brain microvessel are shown viewed from the top (FIGS. 8A, 8D, 8G) or shown in cross-section at either low (FIGS. 8B, 8E, 8H) or high (FIGS. 8C, 8F, 8I) magnification. The rectangles in lower magnifications images of FIGS. 8B, 8E, and 8H indicate respective areas shown at higher magnification of FIGS. 8C, 8F, and 8I, respectively. The fluorescence micrographs show the cell distributions in 3D BBB chips containing brain microvascular endothelium alone (FIGS. 8A-8C, 8J), endothelium with prior plating of brain pericytes on the surface of the gel in the central lumen (FIGS. 8D-8F, 8K), and endothelium with brain astrocytes embedded in the surrounding gel (FIGS. 8G-8I, 8L). High-magnification cross-sections are projections of confocal stacks (bars, 200 μm in FIGS. 8A, 8B, 8D, 8E, 8G, 8H; and bars, 30 μm in FIGS. 8C, 8F, 8I). FIGS. 8D-8I and 8K-8L included F-actin staining 806, FIGS. 8C, 8F, 8I, 8K, and 8L included Hoechst-stained nuclei 802, and FIGS. 8A-8F and 8H-8L included VE-Cadherin staining 804. In FIG. 8G, morphology and intensity masks were used to discriminate astrocytes 806 from endothelial cells 808 A contact point between endothelium and pericytes 810 is shown in FIG. 8F, and a contact point 812 between endothelium or astrocytes is shown in FIG. 81.

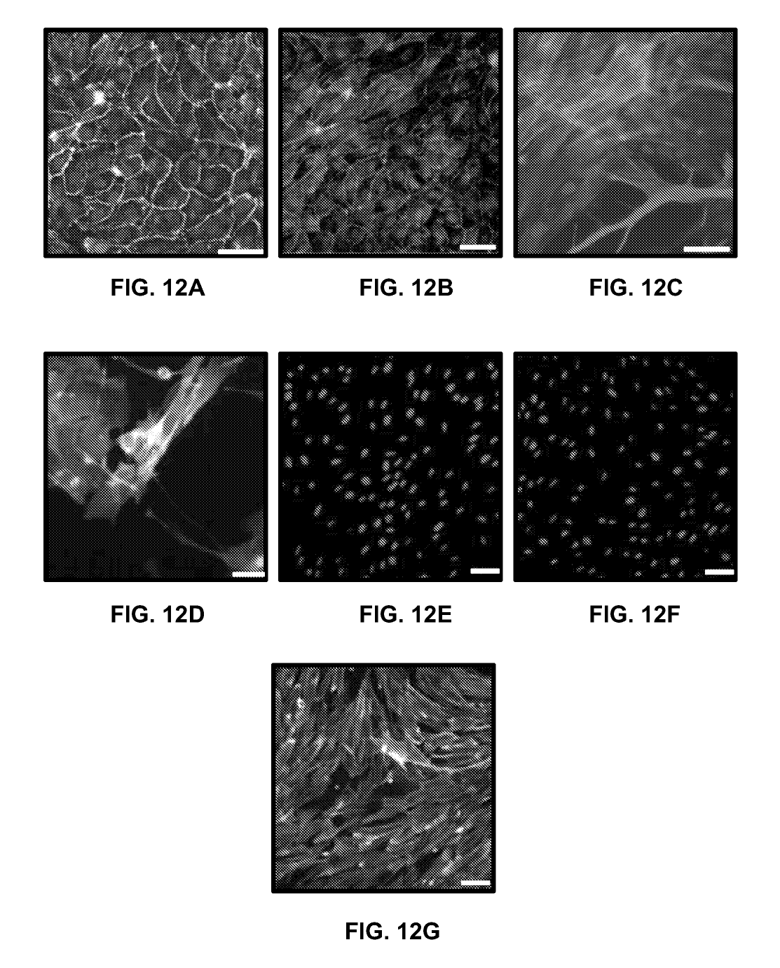

Confocal fluorescence microscopic analysis revealed that the endothelial cells adherent to the inner surface of the collagen gel formed a continuous monolayer with continuous VE-cadherin-containing junctions, thereby creating a cylindrical endothelium-lined microvessel on-chip (FIG. 8A-C). The human brain microvascular endothelial cells also express tight junctions containing ZO-1 protein (FIG. 12). FIGS. 12A-12G illustrate marker expression in human primary cells used to populate a 3D BBB chip according to the embodiments described herein. The continuous endothelium followed the contours of the lumen of the collagen gel, and the endothelial cells secreted their own underlying type IV collagen-containing basement membrane along the cell-matrix interface (FIG. 3) as they do in vivo.

Either primary human brain pericytes or astrocytes that respectively expressed α-smooth muscle actin (SMA) or glial fibrillary acidic protein (GFAP) (FIG. 12) were then integrated into these engineered microvessels. These pericytes do not express endothelial-specific markers (VE-Cadherin and PECAM), nor do they form tight cell-cell junctions that could create a tight permeability barrier of its own, as indicated by the presence of clear spaces between cells (FIG. 12). To explore the contributions of pericytes, they were first seeded onto the luminal surface of the collagen gel for about 30 minutes before plating the endothelial cells, and then maintained them in culture for about 4-5 days. In contrast, the astrocytes were embedded in the gel solution during the viscous fingering process to distribute them throughout the surrounding collagen matrix (FIG. 7C) before the endothelial cells were plated.

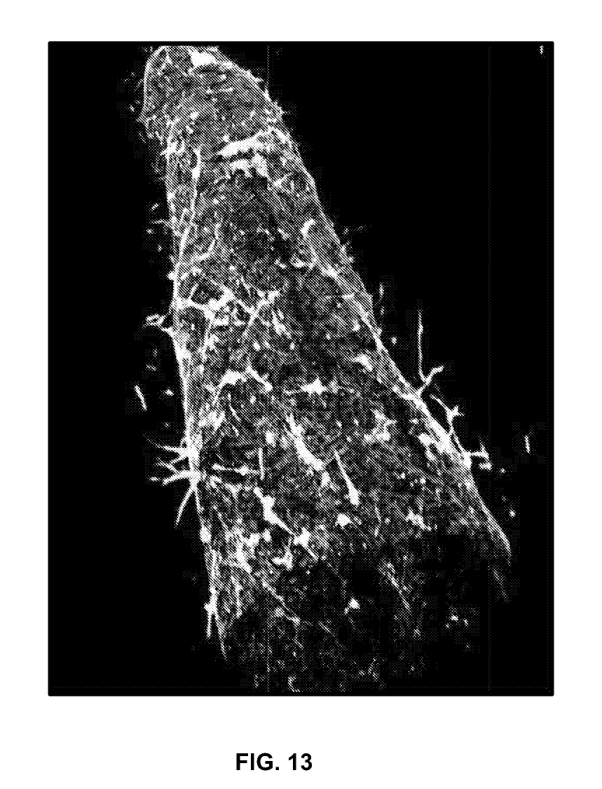

The pericyte seeding method resulted in generally effective integration of the pericytes into the engineered microvessel such that many of them located in a circumferential abluminal distribution in tight association with the basement membrane along the basal surface of the overlying endothelium (FIG. 8D-F and FIG. 13), thus closely mimicking the position they take in vivo. When the astrocytes were embedded in the collagen gels, they filled the ECM space, extended processes towards the endothelium, and contacted the basement membrane at the base of the endothelium (FIG. 8G-I). These cells remained viable and sustained these relationships for the entire about 4-5 day course of the study.

FIGS. 9A-9D illustrate production of an abluminal basement (bar, 100 μm) by brain endothelial cells in a 3D BBB chip according to one embodiment.

Cell Contributions to the Permeability of the Engineered 3D Blood-Brain Barrier

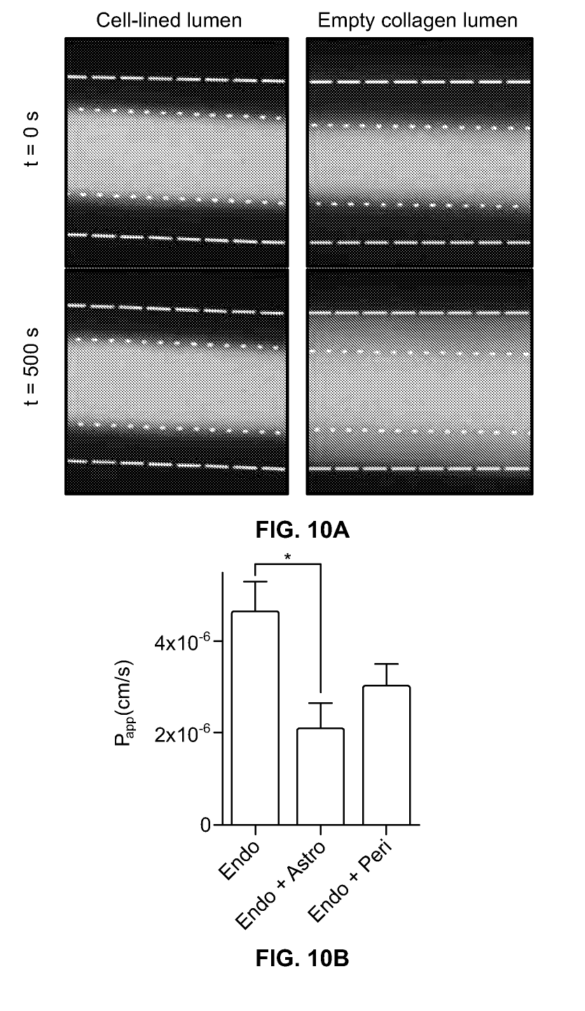

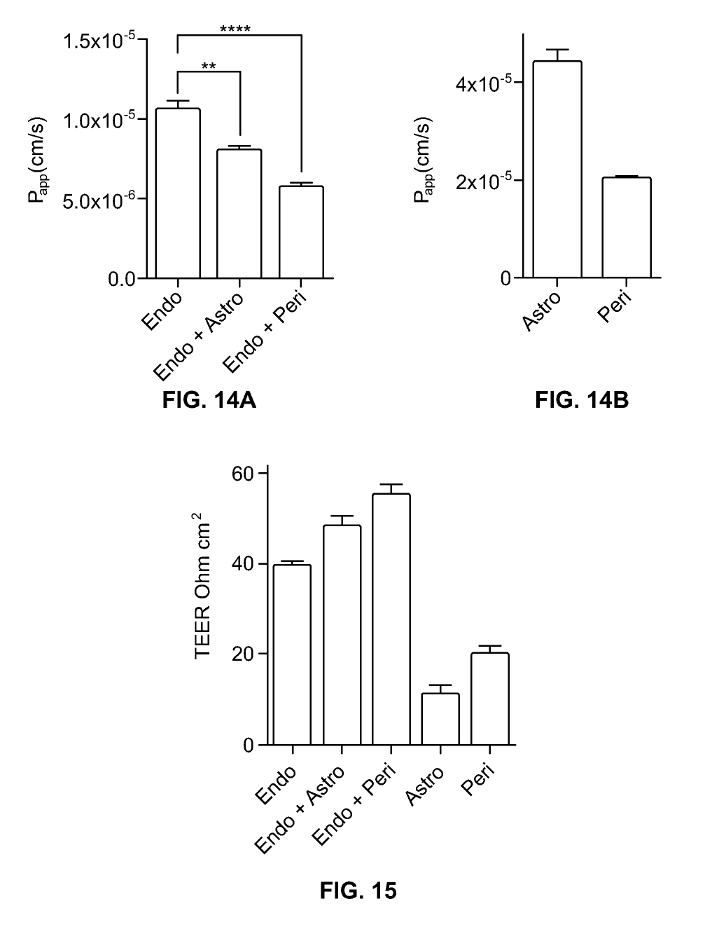

When the paracellular permeability of the engineered microvessel lined only by human brain microvascular endothelium was evaluated by continuously flowing fluorescently-labeled, low molecular weight (3 kDa) dextran through the lumen and analyzing its distribution using time-lapse microscopic imaging, it was found that the presence of the human brain endothelium significantly restricted transfer of the fluorescent probe compared to control microchannels that contained the cylindrical collagen gel without any cells (FIG. 10A). FIGS. 10A, 10B illustrate the establishment of a low permeability barrier by the engineered brain microvascular endothelium in a 3D BBB chip according to one embodiment. In control channels without cells, and in channels that contained pericytes or astrocytes but no endothelium, the fluorescent dextran quickly diffused through the collagen gel and reached the walls of the channel within about 500 seconds, whereas it remained completed restricted to the lumen of the endothelium-lined vessel at this time, which exhibited an apparent permeability of about 4×10⁻⁶ cm/s (FIG. 10A). Importantly, the permeability of the endothelium-lined vessel was reduced even further when either astrocytes or pericytes were co-cultured with the endothelium, with co-cultures synergistically improving barrier function, producing apparent permeabilities in the range of about 2 to 3×10⁻⁶ cm/s (FIG. 10B), which are similar to values previously measured in other in vitro BBB models that have been created with rat, mouse, bovine or immortalized human cells. In contrast, when permeability of monocultures and co-cultures of the same cells cultured in Transwell plates were measured using 3 kDa dextran, values were significantly higher (from about 1×10⁻⁵ to about 6×10⁻⁶ cm/s), indicating that the 3D BBB chip microenvironment promoted improved barrier function in the cultured brain endothelium (FIG. 14A).

Although some breaks in endothelial monolayer continuity and loss of the permeability were observed in some devices, an intact endothelial barrier was observed in over 85% of the chips. Interestingly, cell layers with large defects that were clearly visible in bright-field microscopy showed diffusion similar to bare collagen, whereas cell layers with minor defects could be easily detected due to localized release of the fluorescent tracer, and permeability values in defective monolayer ranged from about 10⁻⁵ to about 10⁻⁴ cm/s.

The cylindrical geometry of the 3D BBB chips did not allow for TEER measurements because it is not possible to introduce electrodes into the lumen without injuring the surrounding cell layers. However, TEER values in the Transwell cultures were measured, which yielded values of about 40-50 Ω×cm² (FIG. 15), that while low, were still within the range that has been previously reported for primary human brain endothelium. The TEER values of monocultures of astrocytes and pericytes were in the higher range of what has been reported in literature; however, these cells do not form a tight monolayer with well-formed intercellular junctions and so this resistance is likely due to the high cell densities in these cultures. In the examples described herein, when endothelial cells were co-cultured with pericytes or astrocytes, the TEER values were higher than those measured in endothelium alone. This increased TEER could be accounted for by adding the TEER values of the individual cell types that were present, as no significant synergistic effect was detected when analyzed by one-way ANOVA. While synergistic effects of astrocytes and pericytes on barrier properties of brain endothelium have been reported previously, it is well known that this response varies greatly depending on cell source and culture conditions, and the conditions of the examples described herein did not support this response.

Taken together, these results show that the 3D BBB chips described herein that were produced with all human primary brain neurovasculature-derived cells display a permeability barrier function that is at least as good as conventional in vitro models of the BBB that use non-human cells or immortalized cells. While there have been studies describing dynamic BBB models with all human primary cells, they did not include a realistic 3D ECM or reconstitute direct cell-cell contacts between the different cell types, as described herein. The examples described herein demonstrate that a parenchymal cell type (human astrocytes) can be incorporated within the ECM surrounding the vessel-like lumen during its formation. Moreover, the sequential seeding of pericytes and endothelial cells resulted in reconstitution of normal tight associations between endothelial cells and pericytes, which has not been observed previously in BBB cultures. In addition, the circular lumen, the development of extended astrocyte cell processes through the 3D collagen matrix, and the direct interaction of perivascular cells and astrocytes with the endothelial monolayer create a culture microenvironment that more closely resembles the in vivo situation, compared to Transwell cultures that are commonly used to model the BBB in vitro.

Finally, the 3D human BBB-on-a-chip was used to study the neuroinflammatory response in vitro. TNF-α is a pro-inflammatory cytokine implicated in various inflammatory diseases of the central nervous system associated with meningitis, multiple sclerosis, Alzheimer's disease, AIDS-related dementia, stroke and brain ischemia, among others. While stimulated macrophages and monocytes are primarily responsible for producing systemic circulating TNF-α, several cell types in the brain, including astrocytes, microglia, and even injured neurons, can secrete TNF-α as a paracrine mediator of inflammation. Elevated TNF-α levels in the brain and serum also have been observed in inflammatory diseases of the central nervous system, such as Alzheimer's disease, multiple sclerosis and traumatic brain injury.

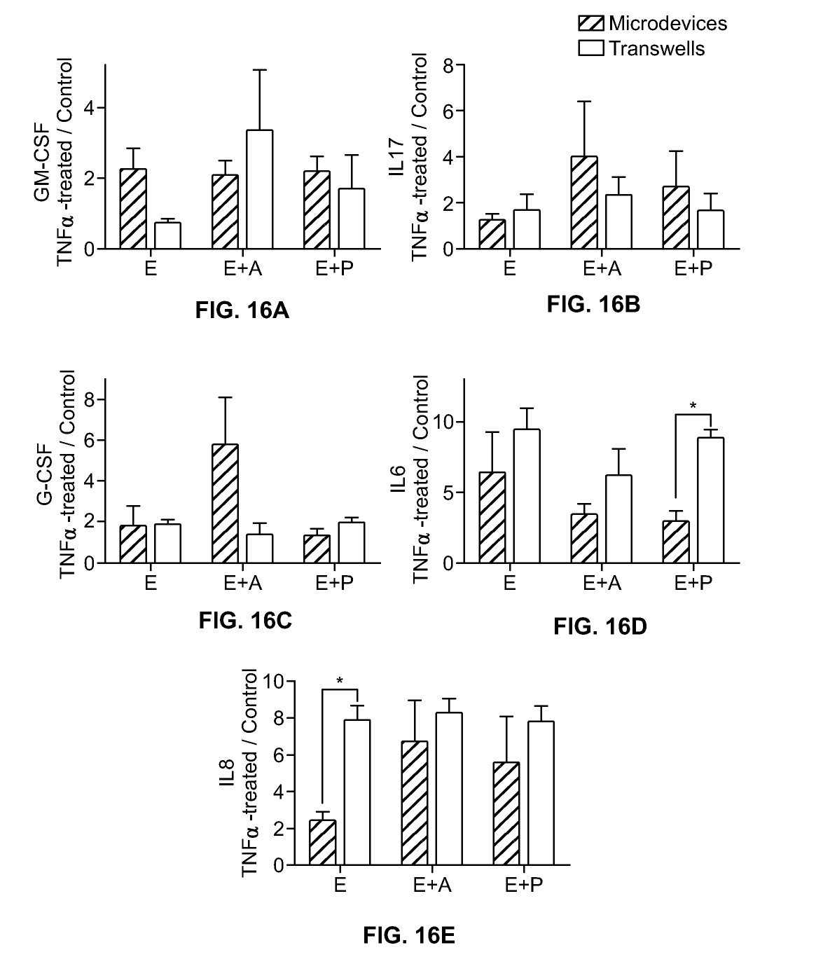

To explore whether we can use the synthetic nature of the 3D BBB chip to analyze the contributions of individual brain vasculature-associated cells to neuroinflammation, the engineered microvessels were cultured in the presence or absence of TNF-α (about 50 ng/ml) that was flowed through the lumen for about 6 hours. Cytokine release profiles produced in the 3D BBB chips containing endothelium with or without either pericytes or astrocytes were then analyzed, and the results were compared to those obtained with similar mono-cultures, as well as co-cultures maintained in commercial Transwell culture plates. Of the seventeen cytokines tested, five exhibited a detectable and generally consistent release pattern in the 3D BBB chips: granulocyte colony stimulating factor (G-CSF), granulocyte macrophage colony stimulating factor (GM-CSF), interleukin-6 (IL-6), interleukin-8 (IL-8/CXCL8), interleukin-17 (IL-17). Comparison of the release profiles of these five cytokines normalized relative to their release from the unstimulated endothelium revealed that secretion of G-CSF and IL-6 was significantly different in 3D BBB chips compared to conventional Transwell co-cultures (FIG. 11A, 11B, FIG. 16).

FIGS. 16A-16E show a comparison of cytokine release profiles after inflammatory stimulation with TNF-α in a microfluidic 3D BBB chip according to the embodiments described herein versus static Transwell cultures. All data represent the levels of cytokines released after TNF-α stimulation normalized to the basal condition for each specific culture. “E” indicates endothelial cells alone, “E+A” indicates a co-culture of endothelial cells and astrocytes, “E+P” indicates a co-culture of endothelial cells and pericytes (* p<0.05 Pairwise Microdevice-Transwell comparison t-tests with Sidak-Bonferroni method for multiple comparisons ; n=4-7 for 3D BBB chips and n=3 for Transwells).

Quantitative comparisons also showed that secretion levels of G-CSF, IL-6 and IL-8 were significantly higher in the microfluidic BBB chip compared to static Transwell cultures, and this difference was most pronounced with G-CSF and IL-6 (FIG. 11C, 11D). Use of the BBB chip also revealed that astrocytes and pericytes can independently enhance the secretion of G-CSF and IL-6 when co-cultured with endothelium even under basal unstimulated conditions, whereas this was not detected in the Transwell system (FIG. 11C, 11D). The fold increase in IL-6 and IL-8 secretion induced by TNF-α was also higher in Transwell cultures than in BBB chips (FIG. 16), which may be partially explained by the higher basal levels of secretion of these cytokines in the chips. In contrast, the induction of G-CSF was more pronounced in 3D BBB chips than in Transwells, and in fact, the levels of this cytokine were almost undetectable in these planar cultures (FIG. 16A-16E).

The ability to detect changes in G-CSF levels in the 3D BBB chip provides a significant advantage over Transwell BBB models for studies on neuroinflammation, as G-CSF is an important neuroprotective cytokine secreted in response to brain injury by endothelial cells, astrocytes, and neurons. G-CSF promotes neuronal survival and proliferation, in addition to stimulating recruitment of bone marrow-derived endothelial progenitor cells that stimulate vascular repair. Animal experiments also have shown that exogenously administered G-CSF can inhibit neuronal cell death after ischemic brain injury. Thus, it is interesting that the examples described herein observed similar strong TNF-α-mediated induction of G-CSF secretion in the 3D co-culture model of brain endothelial cells and astrocytes under microfluidic conditions, whereas this could not be detected when the same cells were co-cultured under static conditions in Transwells. Interestingly, however, because we could independently study the contributions of pericytes and astrocytes to this response, it was discovered that the presence of pericytes was alone sufficient to increase baseline levels of G-CSF secretion in the 3D BBB chip model, and these cultures were generally not sensitive to induction by TNF-α. In contrast, 3D BBB chips containing astrocytes and endothelial cells exhibited up to a 10-fold increase in G-CSF secretion in response to TNF-α stimulation.

FIGS. 11A-D illustrate comparisons of cytokine release profiles after inflammatory stimulation with TNF-α in a microfluidic 3D BBB chip according to the embodiments described herein versus static Transwell cultures. In FIGS. 11A and 11B, all data were normalized to the levels of cytokines released by endothelial cells cultured alone. Concentric scales indicate fold increase.

IL-6, which is strongly expressed by neuronal, glial, and vascular tissue during neuroinflammation in vivo, modulates both the acute and late-stage immune responses. Acutely it prevents neuronal injury by protecting against apoptosis due to oxidative stress and controls the innate immune response that is mediated by neutrophils and monocytes, whereas in later stages of neuroinflammation, IL-6 stimulates angiogenesis and re-vascularization. Levels of secreted IL-6 also correlate with brain infarct size in ischemic stroke and high IL-6 levels are associated with a negative functional outcome after traumatic brain injury. Importantly, a similar response to the inflammatory stimulus TNF-α was observed in the 3D BBB chip co-cultures described herein, with strong IL-6 induction in co-cultures of both astrocytes-endothelial cells and pericytes-endothelial cells (FIGS. 11A, 11C, whereas these responses were barely detectable in Transwell cultures (FIGS. 11B, 11D).

IL-8 is an activating and pro-inflammatory cytokine produced by astrocytes, pericytes, and endothelial cells that is primarily involved in recruiting neutrophils to sites of injury. Levels of IL-8 are markedly increased in the context of neural injury and inhibition of IL-8 signaling is associated with improved outcome in the context of neuroinflammation. While both the 3D BBB chip and Transwell cultures demonstrated enhanced IL-8 production in response to TNF-α stimulation when astrocytes or pericytes were present in combination with endothelial cells, the 3D BBB chip co-cultures again showed a greatly enhanced level of response in terms of the absolute amount of cytokine that was produced (FIGS. 11C, 11D).

Another major difference between the 3D BBB microfluidic chip described herein and Transwell cultures, as well as past microfluidic BBB models, is that these other models contain semi-permeable membranes that separate the interacting cell types. These membranes are typically rigid thick (about 10-50 μm) substrates with pores (about 0.4 −3 μm diameter) that constitute an artificial barrier between the neurovascular cells. In contrast, in the 3D BBB chip, a compliant ECM gel constrained within a confined cylindrical geometry and positioned the endothelial cells, pericytes and astrocytes was utilized in ways that allowed them to reconstitute their normal 3D spatial relationships and reestablish more natural cell-cell interactions, resulting in deposition of an intervening type IV collagen-containing basement membrane. At the same time, it is important to note that the 3D BBB chip does not fully recapitulate the in vivo situation in that the endothelial cells were not subjected to continuous fluid flow and physiologically relevant levels of shear stress during their entire 5 day culture period; however, the cells were exposed to continuous flow when their permeability barrier and neuroinflammatory responses (cytokine secretion profiles) were analyzed. Most previously reported microfluidic models of the BBB similarly fail to include realistic levels of shear stress during sustained culture, probably for similar reasons (e.g., the cost of using large amounts of culture medium).

The lumen of the 3D BBB chip described herein is almost an order of magnitude larger than that of a typical brain microvessel, and the pericytes and astrocytes processes form contacts with a smaller fraction of the endothelium on-chip than in living brain capillaries. However, as all of these features can be controlled and varied in an independent manner using this microengineered approach, it should be possible to determine their relative importance for BBB structure and function in future studies. The data described herein show that this 3D BBB chip reconstitutes more normal spatial relationships and provides a more balanced and physiologically relevant picture of human neurovascular inflammation in vitro than static Transwell cultures, as demonstrated by enhanced secretion of both pro-inflammatory (IL-6) and neuroprotective (G-CSF) cytokines. As the system utilizes all primary human brain-derived cells in addition to mimicking the 3D architecture of the brain microvessel, it also offers an advantage over previously described 2D microfluidic systems that both lacked this structure and utilized non-human or immortalized cells.

This method for establishing co-cultures of multiple types of primary human brain-derived vascular cells (e.g., endothelial cells, pericytes and astrocytes) in microfluidic chips that reconstitute their normal 3D spatial relationships has permitted the dissection of the contributions of these cells to the neuroinflammatory response in vitro. It was first shown that a viscous fingering method can be used to create cylindrical compliant collagen gels within a microfluidic channel, and that hydrostatic pressure-driven flow can be used to control the dimensions of the lumen without having to adjust the channel dimensions or the viscosity of the collagen solution. Using this configuration, multiple modes of co-culture were then established by either embedding astrocytes inside the gel or by performing sequential seeding of pericytes and endothelial cells inside the lumen. The reconstituted all human 3D BBB-on-a-chip formed a permeability barrier similar to that previously reported for cultured non-human or immortalized cells, and the integrity of the endothelium was found to strongly depend on the presence of astrocytes and pericytes in the cultures. Finally, it was demonstrated that the BBB chips that contained these neurovascular cells and reconstituted their normal 3D cell-cell relationships exhibited responses to an inflammatory stimulus (TNF-α) that more closely mimicked those observed in the living brain than the same cells when co-cultured in a planar static Transwell culture. Because this is a synthetic system, additional cell types may be integrated in the 3D BBB chip to create more complex co-cultures in the future, including human immune cells, such as neutrophils, microgli,a and monocytes, as well as human cortical neurons, in addition to the three neurovascular cell types used in the present study. Taken together, these findings suggest that the 3D microfluidic BBB chip described herein may be suitable to study the vascular component of neuroinflammation and other neurological disorders, as well as to help identify new drugs that target these responses.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the claimed invention, which is set forth in the following claims.

1-19. (canceled)

20. A device for simulating a function of a blood-brain-barrier, comprising:
a first structure defining a first chamber, the first chamber comprising a first permeable matrix disposed therein, wherein the first permeable matrix comprises at least one or more lumens each extending therethrough, the at least one or more lumens being lined with at least an endothelial cell layer adherent to the inner surface of the at least one or more lumens; and
a second structure defining a second chamber; the second chamber comprising a first type of brain microenvironment-associated cells distributed therein; and
a membrane located at an interface region between the first chamber and the second chamber to separate the first chamber from the second chamber, the membrane comprising a first side facing toward the first chamber and a second side facing toward the second chamber.

21. The device of claim 20, wherein the first permeable matrix comprises a second type of brain microenvironment-associated cells distributed therein.

22. The device of claim 21, wherein the second type comprises pericytes, astrocytes, microglia, fibroblasts, smooth muscle cells, or a combination of two or more thereof.

23. (canceled)

24. The device of claim 20, wherein the first type of brain microenvironment-associated cells are adhered on the second side of the membrane.

25. The device of claim 20, wherein second chamber further comprises a second permeable matrix disposed therein.

26. The device of claim 25, wherein the first type of brain microenvironment-associated cells are distributed in the second permeable matrix.

27. The device of claim 20, wherein the first type comprises astrocytes, microglia, neurons, or a combination of two or more thereof.

28-44. (canceled)

45. A method of making a device for simulating a function of a tissue comprising:
providing a body comprising:
a first structure defining a first chamber, at least a portion of the first chamber filled with a viscous solution of first matrix molecules disposed therein,
a second structure defining a second chamber, and
a membrane located at an interface region between the first chamber and the second chamber to separate the first chamber from the second chamber, the membrane including a first side facing toward the first chamber and a second side facing toward the second chamber;
flowing at least one pressure-driven fluid with low viscosity through the viscous solution in the first chamber to create one or more lumens each extending through the viscous solution; and
gelling, polymerizing, and/or crosslinking the viscous solution in the first chamber, thereby forming a first permeable matrix comprising one or more lumen(s) each extending therethrough; and
populating at least a portion of the second chamber with tissue specific cells and/or blood vessel-associated cells.

46. The method of claim 45, wherein the populating comprising growing the tissue specific cells of a first type and/or blood vessel-associated cells on the second side of the membrane.

47. The method of claim 45, wherein the populating comprising forming a second permeable matrix disposed in the second chamber, wherein the second permeable matrix comprises the tissue specific cells of a second type.

48-61. (canceled)

62. A method of making a cell-lined lumen in a microfluidic device, the method comprising:
a) providing
i) a microfluidic device comprising a chamber, at least a portion of the chamber filled with a viscous solution of first matrix molecules, and
ii) cells;
b) flowing a fluid through the viscous solution, said fluid having a lower viscosity than said viscous solution;
c) gelling, polymerizing, and/or crosslinking the viscous solution so as to form a lumen comprising a permeable matrix; and
d) introducing said cells into said lumen so as to create a cell-lined lumen.

63. The method of claim 62, wherein said cells are human cerebral endothelial cells lining the lumen.

64. The method of claim 63, wherein said endothelial cells are co-cultured with astrocytes.

65. The device of claim 20, wherein the endothelial cell layer expresses VE-cadherin at an intercellular adherens junction.
